(19) United States
(12) Patent Application Publication
(10) Pub. No.: US 2015/0017239 A1
(43) Pub. Date: Jan. 15, 2015

(54) METHOD FOR TREATING INTESTINAL DISEASES PRESENTING AT LEAST ONE INFLAMMATORY COMPONENT

(71) Applicants: COSMO TECHNOLOGIES LIMITED, Dublin (IE); SANTARUS INC., San Diego, CA (US)

(72) Inventors: Luigi Moro, Lainate (MI) (IT); Gerald Thomas Proehl, San Diego, CA (US); Wendell Wierenga, Rancho Santa Fe, CA (US); Michael Fangching Huang, Cerritos, CA (US); Emerson David Ballard, II, Carlsbad, CA (US)

(73) Assignees: SANTARUS INC., San Diego, CA (US); COSMO TECHNOLOGIES LIMITED, Dublin (IE)

(21) Appl. No.: 14/378,114

(22) PCT Filed: Feb. 13, 2013
(86) PCT No.: PCT/EP2013/052838

§ 371 Date: Aug. 12, 2014

Related U.S. Application Data
(60) Provisional application No. 61/598,308, filed on Feb. 13, 2012.

(51) Int. Cl. A61K 9/20; A61K 9/28; A61K 31/58

(52) U.S. Cl. CPC A61K 9/2013; A61K 31/58; A61K 9/2846; A61K 9/2054; A61K 9/2009
USPC 424/465; 514/174

(57) ABSTRACT

The present disclosure relates to methods for treating intestinal diseases presenting at least one inflammatory component such as inflammatory bowel disease or diverticular disease and/or maintaining remission of intestinal diseases presenting at least one inflammatory component such as inflammatory bowel disease (IBD) or diverticular disease using budesonide MMX compositions.

### CROSS-REFERENCE TO RELATED APPLICATION

The present application claims priority to U.S. provisional patent application Ser. No. 61/598,308 filed on Feb. 13, 2012. This application is incorporated herein in its entirety.

The present disclosure relates to methods for treating intestinal diseases presenting at least one inflammatory component such as inflammatory bowel disease and/or maintaining remission of intestinal diseases presenting at least one inflammatory component such as inflammatory bowel disease (IBD) using budesonide MMX compositions.

Systemic corticosteroids (SCS) are effective for the short-term induction of remission in active inflammatory bowel disease (IBD), including Ulcerative Colitis (UC) and Crohn's Disease. However, side effects are frequent and the risks associated with long term therapy outweigh the benefits of maintenance treatment. Therefore, alternative agents with fewer side effects are needed. Budesonide, a non-systemic corticosteroid (NSCS) with high first-pass metabolism, combined with MMX® technology, which is designed to deliver active drug to the colon, may be a viable therapeutic option.

Oral dosage forms of budesonide utilizing the MMX® multi-matrix system technology are designed to provide the controlled release and distribution of budesonide throughout the length of the colon. The MMX multi-matrix system technology is described in U.S. Pat. Nos. 7,431,943, 7,410,651, RE43,799, and 8,293,273, and U.S. Patent Application Publication No. 2012/0021052, all of which are hereby incorporated by reference in their entireties for all that they disclose. The dosage forms have topical anti-inflammatory activity and due to an extended first pass effect, have less systemic absorption than other corticosteroids.

Provided herein are methods for treating an intestinal disease presenting at least one inflammatory component and/or maintaining remission of an intestinal disease presenting at least one inflammatory component in a patient previously or simultaneously administered a first composition for treatment of said disease, comprising administering to said patient a second composition comprising a tablet comprising budesonide in an amount effective for treating an intestinal disease presenting at least one inflammatory component and/or maintaining remission of an intestinal disease presenting at least one inflammatory component. In some embodiments, an intestinal disease presenting at least one inflammatory component is a chronic disease. In some embodiments, an intestinal disease presenting at least one inflammatory component is inflammatory bowel disease. In one embodiment, the inflammatory bowel disease is ulcerative colitis. In another embodiment, the ulcerative colitis is active mild to moderate ulcerative colitis. In a further embodiment, the inflammatory bowel disease is Crohn's disease. In some embodiments, the intestinal disease presenting at least one inflammatory component may present an infective component. In one embodiment, the inflammatory bowel disease which may present an infective component is ulcerative colitis. In one embodiment, the inflammatory bowel disease which may present an infective component is acute diverticulitis. In a further embodiment, the inflammatory bowel disease which may present an infective component is Crohn's disease. In some embodiments, an intestinal disease presenting at least one inflammatory component is an acute disease or an acute phase of an intestinal disease. In one embodiment, the acute disease is a diverticulitis, acute diverticulitis or uncomplicated diverticulitis. In one embodiment, the first composition is administered previously or prior to the second composition. In another embodiment, the first composition is administered simultaneously with the second composition. In another embodiment, the first composition is administered after the second composition.

Also provided herein is a second composition for treating an intestinal disease presenting at least one inflammatory component and/or maintaining remission of an intestinal disease presenting at least one inflammatory component in a patient previously or simultaneously treated with a first composition for treating said disease. In one embodiment, the second composition comprises budesonide in an amount effective for treating an intestinal disease presenting at least one inflammatory component and/or maintaining remission of an intestinal disease presenting at least one inflammatory component. In some embodiments, an intestinal disease presenting at least one inflammatory component is a chronic disease. In some embodiments, an intestinal disease presenting at least one inflammatory component is inflammatory bowel disease. In one embodiment, the inflammatory bowel disease is ulcerative colitis. In another embodiment, the ulcerative colitis is active mild to moderate ulcerative colitis. In a further embodiment, the inflammatory bowel disease is Crohn's disease. In some embodiments, the intestinal disease presenting at least one inflammatory component may present an infective component. In one embodiment, the inflammatory bowel disease which may present an infective component is ulcerative colitis. In one embodiment, the inflammatory bowel disease which may present an infective component is acute diverticulitis. In a further embodiment, the inflammatory bowel disease which may present an infective component is Crohn's disease. In some embodiments, an intestinal disease presenting at least one inflammatory component is an acute disease or an acute phase of an intestinal disease. In one embodiment, the acute disease is acute diverticulitis. In one embodiment, the patient is treated with the first composition previously to the second composition. In another embodiment, the patient is treated with the first composition simultaneously with the second composition.

In some embodiments, the second composition comprises a tablet comprising about 1 mg to about 12 mg of budesonide. In other embodiments, the second composition comprises a tablet comprising about 1 mg to about 18 mg budesonide. In another embodiment, the second composition comprises a tablet comprising about 3 mg budesonide. In another embodiment, the second composition comprises a tablet comprising about 4.5 mg budesonide. In another embodiment, the second composition comprises a tablet comprising about 6 mg budesonide. In another embodiment, the second composition comprises a tablet comprising about 9 mg budesonide. In another embodiment, the second composition comprises a tablet comprising about 12 mg budesonide. In another embodiment, the second composition comprises a tablet comprising about 15 mg budesonide. In another embodiment, the second composition comprises a tablet comprising about 18 mg budesonide.

In some embodiments, the second composition comprises about 1 mg to 18 mg budesonide as a single dose or as a divided dose. In one embodiment, the second composition comprises about 1 mg to 12 mg budesonide as a single dose or as a divided dose. In another embodiment, the second composition comprises about 18 mg budesonide as a single dose or as a divided dose. In another embodiment, the second composition comprises about 15 mg budesonide as a single dose or as a divided dose. In another embodiment, the second composition comprises about 12 mg budesonide as a single dose or as a divided dose. In another embodiment, the second composition comprises about 9 mg budesonide as a single dose or as a divided dose. In another embodiment, the second composition comprises about 6 mg budesonide as a single dose or as a divided dose. In another embodiment, the second composition comprises about 4.5 mg budesonide as a single dose or as a divided dose. In another embodiment, the second composition comprises about 3 mg budesonide as a single dose or as a divided dose.

In one embodiment, the first and second compositions are administered to a patient sequentially with some period of time between the two administrations. In another embodiment, the first and second compositions are administered to the patient at the same time. In another embodiment, the first and second compositions are administered to the patient either sequentially or at the same time for a period of up to 8 weeks or a portion thereof. In another embodiment, the first and second compositions are administered to the patient either sequentially or at the same time for a period of 8 weeks or a portion thereof, such as a few days or 4 weeks, 7.5 weeks and the like. In another embodiment, the first and second compositions are administered to the patient either sequentially or at the same time for a period of up to 8 weeks or portion thereof and the second composition is administered to the patient once daily. In another embodiment, the first and second compositions are administered to the patient either sequentially or at the same time for a period of up to 8 weeks or a portion thereof and the second composition is administered to the patient once daily in the morning with or without food. In some embodiments, the second composition is administered for at least 12 months. In one embodiment, the second composition comprises about 1 mg to 12 mg budesonide as a single dose or as a divided dose. In another embodiment, the second composition comprises about 18 mg budesonide as a single dose or as a divided dose. In another embodiment, the second composition comprises about 15 mg budesonide as a single dose or as a divided dose. In another embodiment, the second composition comprises about 12 mg budesonide as a single dose or as a divided dose. In another embodiment, the second composition comprises about 9 mg budesonide as a single dose or as a divided dose. In another embodiment, the second composition comprises about 6 mg budesonide as a single dose or as a divided dose. In another embodiment, the second composition comprises about 4.5 mg budesonide as a single dose or as a divided dose. In another embodiment, the second composition comprises about 3 mg budesonide as a single dose or as a divided dose.

Provided herein in some embodiments are methods for treating an intestinal disease presenting at least one inflammatory component and/or maintaining remission of an intestinal disease presenting at least one inflammatory component in a patient previously administered a first composition for treatment of said disease, comprising administering to said patient a second composition comprising: (1) a tablet core comprising: (a) budesonide in an amount effective for treating an intestinal disease presenting at least one inflammatory component and/or maintaining remission of an intestinal disease presenting at least one inflammatory component, (b) at least one lipophilic excipient; (c) at least one amphiphilic excipient; and (d) at least one hydrophilic excipient; and (2) at least one coating on said tablet core, said coating comprising a gastro-resistant film.

Also provided herein is a second composition for treating an intestinal disease presenting at least one inflammatory component and/or maintaining remission of an intestinal disease presenting at least one inflammatory component in a patient previously or simultaneously treated with a first composition for treating said disease. In one embodiment, the second composition comprises a tablet core and a coating on the tablet core. In one embodiment, the tablet core comprises (a) budesonide in an amount effective for treating or maintaining remission of an intestinal disease presenting at least one inflammatory component, (b) at least one lipophilic excipient, (c) at least one amphiphilic excipient and (d) at least one hydrophilic excipient. In one embodiment, the coating comprises a gastro-resistant film.

Provided herein in some embodiments are methods for treating inflammatory bowel disease and/or maintaining remission of inflammatory bowel disease in a patient previously administered a first composition for treatment of said disease, comprising administering to said patient a second composition comprising: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) at least one coating on said tablet core, said coating comprising a gastro-resistant film.

Also provided herein is a second composition for treating inflammatory bowel disease and/or maintaining remission of inflammatory bowel disease in a patient previously treated with a first composition for treating said disease. In one embodiment, the second composition comprises a tablet core and a coating on the tablet core. In one embodiment, the tablet core comprises (a) budesonide in an amount effective for treating or maintaining remission of an intestinal disease presenting at least one inflammatory component, (b) at least one lipophilic excipient, (c) at least one amphiphilic excipient and (d) at least one hydrophilic excipient. In one embodiment, the coating comprises a gastro-resistant film.

Provided herein in some embodiments are methods for treating an intestinal disease presenting at least one inflammatory component and/or maintaining remission of an intestinal disease presenting at least one inflammatory component in a patient previously administered a first composition for treatment of said disease, comprising administering to said patient a second composition comprising: (1) a multi-matrix tablet core comprising: (a) budesonide in an amount effective for treating an intestinal disease presenting at least one inflammatory component and/or maintaining remission of an intestinal disease presenting at least one inflammatory component, (b) at least one lipophilic matrix-forming excipient; (c) at least one amphiphilic matrix-forming excipient; and (d) at least one hydrophilic matrix-forming excipient; and (2) at least one coating on said multi-matrix tablet core, said coating comprising a gastro-resistant film.

Also provided herein is a second composition for treating an intestinal disease presenting at least one inflammatory component and/or maintaining remission of an intestinal disease presenting at least one inflammatory component in a patient previously treated with a first composition for treatment of said disease. In one embodiment, the second composition comprises: (1) a multi-matrix tablet core comprising: (a) budesonide in an amount effective for treating an intestinal disease presenting at least one inflammatory component and/or maintaining remission of an intestinal disease presenting at least one inflammatory component, (b) at least one lipophilic matrix-forming excipient; (c) at least one amphiphilic matrix-forming excipient; and (d) at least one hydrophilic matrix-forming excipient; and (2) at least one coating on said multi-matrix tablet core, said coating comprising a gastro-resistant film.

In one embodiment, the second composition comprises budesonide and is administered to patients for the induction of remission in patients with active, mild to moderate ulcerative colitis. In another embodiment, the second composition comprises budesonide and is administered to patients for the induction of remission in patients with active, mild to moderate ulcerative colitis with an inadequate response to the administration of a first composition comprising mesalamine. In some embodiments, the second composition comprises about 1 mg to about 12 mg budesonide. In other embodiments, the second composition comprises about 1 mg to about 18 mg budesonide. In one embodiment, the second composition comprises about 18 mg budesonide. In another embodiment, the second composition comprises about 15 mg budesonide. In another embodiment, the second composition comprises about 12 mg budesonide. In another embodiment, the second composition comprises about 9 mg budesonide. In another embodiment, the second composition comprises about 6 mg budesonide. In another embodiment, the second composition comprises about 4.5 mg budesonide. In another embodiment, the second composition comprises about 3 mg budesonide. The amount of budesonide may be in a single dose or in a divided dose.

In one embodiment, the second composition comprises about 9 mg budesonide. In another embodiment, the second composition comprises about 6 mg budesonide. In another embodiment, the second composition comprises about 4.5 mg budesonide. In another embodiment, the second composition comprises about 3 mg budesonide. The amount of budesonide may be in a single dose or in a divided dose.

In some embodiments, the second composition comprises about 1 mg to about 18 mg budesonide. In another embodiment, the second composition comprises about 18 mg budesonide. In another embodiment, the second composition comprises about 15 mg budesonide. In another embodiment, the second composition comprises about 12 mg budesonide. In another embodiment, the second composition comprises about 9 mg budesonide. In another embodiment, the second composition comprises about 6 mg budesonide. In another embodiment, the second composition comprises about 4.5 mg budesonide. In another embodiment, the second composition comprises about 3 mg budesonide. The amount of budesonide may be in a single dose or in a divided dose.

In one embodiment, the gastro-resistant film comprising the second composition begins to dissolve or break down at a pH equal to or greater than 7. In another embodiment, the gastro-resistant film comprising the second composition begins to dissolve at a pH equal to or greater than 7.2.

In another embodiment, the second composition comprises a delayed and extended-release tablet, comprising budesonide. In another embodiment, the delayed and extended-release tablet is coated with a polymer film, which breaks down or dissolves at or above pH 7.0. In another embodiment, the delayed and extended-release tablet comprises about 1 mg to about 12 mg budesonide or about 1 mg to about 18 mg of budesonide. In one embodiment, the delayed and extended-release tablet comprises about 3.0 mg budesonide. In another embodiment, the delayed and extended-release tablet comprises about 4.5 mg budesonide. In another embodiment, the delayed and extended-release tablet comprises about 6 mg budesonide. In another embodiment, the delayed and extended-release tablet comprises about 9 mg budesonide. In another embodiment, the delayed and extended-release tablet comprises about 12 mg budesonide. In another embodiment, the delayed and extended-release tablet comprises about 15 mg budesonide. In another embodiment, the delayed and extended-release tablet comprises about 18 mg budesonide.

In one embodiment, the second composition comprises a delayed and extended-release tablet, comprising budesonide and further comprises a compound selected from stearic acid, lecithin, microcrystalline cellulose, hydroxypropylcellulose, lactose, silicon dioxide, magnesium stearate, methacrylic acid copolymer types A and B, talc, triethylcitrate, and titanium dioxide. In another embodiment, the second composition comprises a delayed and extended-release tablet, comprising budesonide and further comprising at least one compound selected from stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In one embodiment, the second composition delivers budesonide selectively in the lower portion of the gastrointestinal tract of a patient to which it is administered. In another embodiment, the second composition delivers budesonide selectively in the colon of a patient to which it is administered.

In one embodiment, the oral administration of a second composition comprising about 9 mg budesonide to a patient results in a peak plasma concentration (Cmax) of budesonide in the patient of about 1.35±0.96 ng/mL. In another embodiment, the oral administration of a second composition comprising about 9 mg budesonide to a patient results in a time to peak concentration (Tmax) of budesonide in the patient of about 13.3±5.9 hours. In another embodiment, the oral administration of a second composition comprising about 9 mg budesonide to a patient results in an area under the plasma concentration time curve (AUC) of budesonide in the patient of about 16.43±10.52 ng·hr/mL. In another embodiment, the oral administration of a second composition comprising about 9 mg budesonide to a patient under fasting conditions or with a high-fat meal results in a Cmax of budesonide in a patient that is, relative to fasting conditions, decreased by 27% under high-fat meal conditions while, relative to fasting conditions, there is no significant decrease in AUC under high-fat meal conditions. In another embodiment, the oral administration of a second composition comprising about 9 mg budesonide to a patient under fed conditions results in a T_{lag} of budesonide in the patient of about 9.8±3.6 hours. In another embodiment, the oral administration of a second composition comprising about 9 mg budesonide to a patient under fasting conditions results in a T_{lag} of budesonide in the patient of about 7.4±4.2 hours.

Also provided are methods for treating an intestinal disease presenting at least one inflammatory component and/or maintaining remission of an intestinal disease presenting at least one inflammatory component in a patient previously administered a first composition for treating said disease, comprising administering to said patient a second composition comprising: a tablet core comprising 1 mg to 18 mg budesonide, stearic acid, lecithin, and hydroxypropylcellulose; and a coating on said tablet core, said coating comprising a gastro-resistant film which comprises at least one methacrylic acid polymer or copolymer. In one embodiment, the amount of budesonide is 18 mg. In another embodiment, amount of budesonide is 15 mg. In another embodiment, the amount of budesonide is 12 mg. In another embodiment, the amount of budesonide is 9 mg. In another embodiment, the amount of budesonide is 6 mg. In another embodiment, the amount of budesonide is 4.5 mg. In another embodiment, the amount of budesonide is 3 mg. In some embodiments, the tablet core is a multi-matrix tablet core.

Also provided is a second composition for treating an intestinal disease presenting at least one inflammatory component and/or maintaining remission of an intestinal disease presenting at least one inflammatory component in a patient previously treated with a first composition for treating said disease. In one embodiment, the second composition comprises a tablet core comprising 1 mg to 18 mg budesonide, stearic acid, lecithin, and hydroxypropylcellulose; and a coating on the tablet core. In one embodiment the coating comprises a gastro-resistant film which comprises at least one methacrylic acid polymer or copolymer. In one embodiment, the amount of budesonide is 18 mg. In another embodiment, amount of budesonide is 15 mg. In another embodiment, the amount of budesonide is 12 mg. In another embodiment, the amount of budesonide is 9 mg. In another embodiment, the amount of budesonide is 6 mg. In another embodiment, the amount of budesonide is 4.5 mg. In another embodiment, the amount of budesonide is 3 mg. In some embodiments, the tablet core is a multi-matrix tablet core.

Also provided are methods for treating inflammatory bowel disease and/or maintaining remission of inflammatory bowel disease in a patient previously administered a first composition for treating said disease, comprising administering to said patient a second composition comprising: a tablet core comprising 9 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided is a second composition for treating inflammatory bowel disease and/or maintaining remission of inflammatory bowel disease in a patient previously administered a first composition for treating said disease, comprising administering to said patient a second composition comprising: a tablet core comprising 9 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided are methods for treating inflammatory bowel disease and/or maintaining remission of inflammatory bowel disease in a patient previously administered a first composition for treating said disease, comprising administering to said patient a second composition comprising: a tablet core comprising 6 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided is a second composition for treating inflammatory bowel disease and/or maintaining remission of inflammatory bowel disease in a patient previously administered a first composition for treating said disease, comprising administering to said patient a second composition comprising: a tablet core comprising 6 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided are methods for treating inflammatory bowel disease and/or maintaining remission of inflammatory bowel disease in a patient previously administered a first composition for treating said disease, comprising administering to said patient a second composition comprising: a tablet core comprising 4.5 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided is a second composition for treating inflammatory bowel disease and/or maintaining remission of inflammatory bowel disease in a patient previously administered a first composition for treating said disease, comprising administering to said patient a second composition comprising: a tablet core comprising 4.5 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided are methods for treating inflammatory bowel disease and/or maintaining remission of inflammatory bowel disease in a patient previously administered a first composition for treating said disease, comprising administering to said patient a second composition comprising: a tablet core comprising 3 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided is a second composition for treating inflammatory bowel disease and/or maintaining remission of inflammatory bowel disease in a patient previously administered a first composition for treating said disease, comprising administering to said patient a second composition comprising: a tablet core comprising 3 mg budesonide, stearic acid, lecithin, and hydroxypropylcellulose; and a coating on said tablet core, said coating comprising a gastro-resistant film which comprises at least one methacrylic acid polymer or copolymer. In some embodiments, the tablet core is a multi-matrix tablet core.

Also provided are methods for treating an intestinal disease presenting at least one inflammatory component and/or maintaining remission of an intestinal disease presenting at least one inflammatory component in a patient previously administered a first composition for treating said disease, comprising administering to a patient in need thereof a second composition comprising: (1) budesonide in an amount effective for treating an intestinal disease presenting at least one inflammatory component and/or maintaining remission of an intestinal disease presenting at least one inflammatory component, (2) means for topically delivering in the gastrointestinal tract said effective amount of budesonide.

Also provided are methods for treating inflammatory bowel disease and/or maintaining remission of inflammatory bowel disease in a patient previously administered a first composition for treating said disease, comprising administering to a patient in need thereof a second composition comprising: (1) budesonide in an amount effective for treating inflammatory bowel disease and/or maintaining remission of inflammatory bowel disease, (2) means for topically delivering in the gastrointestinal tract said effective amount of budesonide.

Also provided herein are methods for treating an intestinal disease presenting at least one inflammatory component and/or maintaining remission of an intestinal disease presenting at least one inflammatory component in a patient simultaneously administered a first composition for treatment of said disease comprising administering to said patient a second composition comprising: (1) a tablet core comprising: (a) budesonide in an amount effective for treating an intestinal disease presenting at least one inflammatory component and/or maintaining remission of an intestinal disease presenting at least one inflammatory component, (b) at least one lipophilic excipient; (c) at least one amphiphilic excipient; and (d) at least one hydrophilic excipient; and (2) a coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein is a second composition for treating an intestinal disease presenting at least one inflammatory component and/or maintaining remission of an intestinal disease presenting at least one inflammatory component in a patient simultaneously treated with a first composition for treatment of said disease. In one embodiment, the second composition comprises: (1) a tablet core comprising: (a) budesonide in an amount effective for treating an intestinal disease presenting at least one inflammatory component and/or maintaining remission of an intestinal disease presenting at least one inflammatory component, (b) at least one lipophilic excipient; (c) at least one amphiphilic excipient; and (d) at least one hydrophilic excipient; and (2) a coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein are methods for treating inflammatory bowel disease and/or maintaining remission of inflammatory bowel disease in a patient simultaneously administered a first composition for treatment of said disease comprising administering to said patient a second composition comprising: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) a coating on said tablet core, said coating comprising a gastro-resistant film.

Also provided herein is a second composition for treating inflammatory bowel disease and/or maintaining remission of inflammatory bowel disease in a patient simultaneously treated with a first composition for treatment of said disease. In one embodiment, the second composition comprises: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) a coating on said tablet core, said coating comprising a gastro-resistant film.

Also provided are methods for treating an intestinal disease presenting at least one inflammatory component and/or maintaining remission of an intestinal disease presenting at least one inflammatory component in a patient simultaneously administered a first composition for treating said disease, comprising administering to said patient a second composition comprising: a tablet core comprising 1 mg to 18 mg budesonide, stearic acid, lecithin, and hydroxypropylcellulose; and a coating on said tablet core, said coating comprising a gastro-resistant film which comprises at least one methacrylic acid polymer or copolymer. In one embodiment, the amount of budesonide is 18 mg. In another embodiment, amount of budesonide is 15 mg. In another embodiment, the amount of budesonide is 12 mg. In another embodiment, the amount of budesonide is 9 mg. In another embodiment, the amount of budesonide is 6 mg. In another embodiment, the amount of budesonide is 4.5 mg. In another embodiment, the amount of budesonide is 3 mg. In some embodiments, the tablet core is a multi-matrix tablet core.

Also provided is a second composition for treating an intestinal disease presenting at least one inflammatory component and/or maintaining remission of an intestinal disease presenting at least one inflammatory component in a patient simultaneously treated with a first composition for treating said disease. In one embodiment, the second composition comprises: a tablet core comprising 1 mg to 18 mg budesonide, stearic acid, lecithin, and hydroxypropylcellulose; and a coating on said tablet core, said coating comprising a gastro-resistant film which comprises at least one methacrylic acid polymer or copolymer. In one embodiment, the amount of budesonide is 18 mg. In another embodiment, amount of budesonide is 15 mg. In another embodiment, the amount of budesonide is 12 mg. In another embodiment, the amount of budesonide is 9 mg. In another embodiment, the amount of budesonide is 6 mg. In another embodiment, the amount of budesonide is 4.5 mg. In another embodiment, the amount of budesonide is 3 mg. In some embodiments, the tablet core is a multi-matrix tablet core.

Also provided are methods for treating inflammatory bowel disease and/or maintaining remission of inflammatory bowel disease in a patient simultaneously administered a first composition for treatment of said disease, comprising administering to said patient a second composition comprising: a tablet core comprising 9 mg budesonide, stearic acid, lecithin, and hydroxypropylcellulose; and a coating on said tablet core, said coating comprising a gastro-resistant film which comprises at least one methacrylic acid polymer or copolymer.

Also provided is a second composition for treating inflammatory bowel disease and/or maintaining remission of inflammatory bowel disease in a patient simultaneously treated with a first composition for treatment of said disease. In one embodiment, the second composition comprising: a tablet core comprising 9 mg budesonide, stearic acid, lecithin, and hydroxypropylcellulose; and a coating on said tablet core, said coating comprising a gastro-resistant film which comprises at least one methacrylic acid polymer or copolymer.

Also provided are methods for treating inflammatory bowel disease and/or maintaining remission of inflammatory bowel disease in a patient simultaneously administered a first composition for treatment of said disease, comprising administering to said patient a second composition comprising: a tablet core comprising 6 mg budesonide, stearic acid, lecithin, and hydroxypropylcellulose; and a coating on said tablet core, said coating comprising a gastro-resistant film which comprises at least one methacrylic acid polymer or copolymer.

Also provided is a second composition for treating inflammatory bowel disease and/or maintaining remission of inflammatory bowel disease in a patient simultaneously treated with a first composition for treatment of said disease. In one embodiment, the second composition comprising: a tablet core comprising 6 mg budesonide, stearic acid, lecithin, and hydroxypropylcellulose; and a coating on said tablet core, said coating comprising a gastro-resistant film which comprises at least one methacrylic acid polymer or copolymer.

Also provided are methods for treating inflammatory bowel disease and/or maintaining remission of inflammatory bowel disease in a patient simultaneously administered a first composition for treatment of said disease, comprising administering to said patient a second composition comprising: a tablet core comprising 4.5 mg budesonide, stearic acid, lecithin, and hydroxypropylcellulose; and a coating on said tablet core, said coating comprising a gastro-resistant film which comprises at least one methacrylic acid polymer or copolymer.

Also provided is a second composition for treating inflammatory bowel disease and/or maintaining remission of inflammatory bowel disease in a patient simultaneously treated with a first composition for treatment of said disease. In one embodiment, the second composition comprising: a tablet core comprising 4.5 mg budesonide, stearic acid, lecithin, and hydroxypropylcellulose; and a coating on said tablet core, said coating comprising a gastro-resistant film which comprises at least one methacrylic acid polymer or copolymer.

Also provided are methods for treating inflammatory bowel disease and/or maintaining remission of inflammatory bowel disease in a patient simultaneously administered a first composition for treatment of said disease, comprising administering to said patient a second composition comprising: a tablet core comprising 3 mg budesonide, stearic acid, lecithin, and hydroxypropylcellulose; and a coating on said tablet core, said coating comprising a gastro-resistant film which comprises at least one methacrylic acid polymer or copolymer.

Also provided is a second composition for treating inflammatory bowel disease and/or maintaining remission of inflammatory bowel disease in a patient simultaneously treated with a first composition for treatment of said disease. In one embodiment, the second composition comprising: a tablet core comprising 3 mg budesonide, stearic acid, lecithin, and hydroxypropylcellulose; and a coating on said tablet core, said coating comprising a gastro-resistant film which comprises at least one methacrylic acid polymer or copolymer.

Also provided are methods for treating inflammatory bowel disease and/or maintaining remission of inflammatory bowel disease in a patient simultaneously administered a first composition for treatment of said disease, comprising administering to a said patient in need thereof a second composition comprising: (1) budesonide in an amount effective for treating or maintaining remission of inflammatory bowel disease, (2) means for topically delivering in the gastrointestinal tract said effective amount of budesonide.

Also provided are methods for treating an intestinal disease presenting at least one inflammatory component and/or maintaining remission of an intestinal disease presenting at least one inflammatory component in a patient simultaneously administered a first composition for treatment of said disease, comprising administering to a said patient in need thereof a second composition comprising: (1) budesonide in an amount effective for treating or maintaining remission of an intestinal disease presenting at least one inflammatory component, (2) means for topically delivering in the gastrointestinal tract said effective amount of budesonide.

Also provided are methods for treating ulcerative colitis in a patient in need of such treatment, said patient having been previously administered a composition comprising 5-aminosalicylic acid, said method comprising administering to said patient a second composition in the form of a single tablet comprising about 9 mg of budesonide. It is understood in the description that follows that reference to a UCDAI score of greater than or equal to 4 prior to the treatment would correspond to a CAI score of greater than 4 and that a UCDAI score of less than or equal to 1 would correspond to a CAI score of no more than 4. In one embodiment, the second composition is administered to said patient for up to 8 weeks or a portion thereof. In some embodiments, the patient has a UCDAI score of greater than or equal to 4 prior to said second composition being administered to said patient. In one embodiment, the second composition is administered to said patient once daily for an 8 week period, or up to 8 weeks or a portion thereof. In some embodiments, the UCDAI score for said patient is less than or equal to 1 after the 8 week period or after a treatment period of up to 8 weeks or a portion thereof. In other embodiments, the patient is experiencing an ulcerative colitis flare prior to said second composition being administered to said patient. In one embodiment, the second composition is administered to said patient once daily for an 8 week period, or up to 8 weeks or a portion thereof. In one embodiment, the ulcerative colitis in said patient is in remission after said 8 week period or after a treatment period of up to 8 weeks or a portion thereof.

Also provided is a second composition for treating ulcerative colitis in a patient in need of such treatment, said patient having been previously treated with a composition comprising 5-aminosalicylic acid. In one embodiment, the second composition is in the form of a single tablet comprising about 9 mg of budesonide. In one embodiment, the second composition is administered to said patient for up to 8 weeks or a portion thereof. In some embodiments, the patient has a UCDAI score of greater than or equal to 4 prior to said second composition being administered to said patient. In another embodiment, the second composition is administered to said patient once daily for an 8 week period of for up to 8 weeks or a portion thereof. In some embodiments, the UCDAI score for said patient is less than or equal to 1 after the 8 week period or after a treatment period of up to 8 weeks or a portion thereof. In other embodiments, the patient is experiencing an ulcerative colitis flare prior to said second composition being administered to said patient. In one embodiment, the second composition is administered to said patient once daily for an 8 week period or for up to 8 weeks or a portion thereof. In one embodiment, the ulcerative colitis in said patient is in remission after said 8 week period or after a treatment period of up to 8 weeks or a portion thereof.

Also provided are methods for treating ulcerative colitis in a patient in need of such treatment, said method comprising administering to said patient a first and a second composition, said first composition comprising 5-aminosalicylic acid and said second composition is in the form of a single tablet comprising about 9 mg of budesonide. In one embodiment, the second composition is administered to said patient for up to 8 weeks or a portion thereof. In some embodiments, the patient has a UCDAI score of greater than or equal to 4 prior to said second composition being administered to said patient. In one embodiment, the second composition is administered to said patient once daily for an 8 week period or for up to 8 weeks or a portion thereof. In some embodiments, the UCDAI score for said patient is less than or equal to 1 after the 8 week period or after a treatment period of up to 8 weeks or a portion thereof. In other embodiments, the patient is experiencing an ulcerative colitis flare prior to said second composition being administered to said patient. In one embodiment, the second composition is administered to said patient once daily for an 8 week period or for up to 8 weeks or a portion thereof. In one embodiment, the ulcerative colitis in said patient is in remission after said 8 week period or after a treatment period of up to 8 weeks or a portion thereof. In some embodiments the first composition and second composition are administered to the patient simultaneously.

Also provided are a first composition and a second composition for treating ulcerative colitis in a patient in need of such treatment. In one embodiment, the first composition is 5-aminosalicylic acid. In one embodiment, the second composition is in the form of a single tablet comprising about 9 mg of budesonide. In one embodiment, the second composition is administered to said patient for up to 8 weeks or a portion thereof. In some embodiments, the patient has a UCDAI score of greater than or equal to 4 prior to said second composition being administered to said patient. In another embodiment, the second composition is administered to said patient once daily for an 8 week period or for up to 8 weeks or a portion thereof. In some embodiments, the UCDAI score for said patient is less than or equal to 1 after the 8 week period or after a treatment period of up to 8 weeks or a portion thereof. In other embodiments, the patient is experiencing an ulcerative colitis flare prior to said second composition being administered to said patient. In one embodiment, the second composition is administered to said patient once daily for an 8 week period or for up to 8 weeks or a portion thereof. In one embodiment, the ulcerative colitis in said patient is in remission after said 8 week period or after a treatment period of up to 8 weeks or a portion thereof. In some embodiments the first composition and second composition are administered to the patient simultaneously.

Also provided are methods for treating ulcerative colitis in a patient in need of such treatment, said patient having been previously administered a composition comprising 5-aminosalicylic acid, said method comprising administering to said patient a first composition and a second composition, said first composition comprising 5-aminosalicylic acid and said second composition is in the form of a single tablet comprising about 9 mg of budesonide. In one embodiment, the second composition is administered to said patient for up to 8 weeks or a portion thereof. In some embodiments, the patient has a UCDAI score of greater than or equal to 4 prior to said second composition being administered to said patient. In another embodiment, the second composition is administered to said patient once daily for an 8 week period or for up to 8 weeks or a portion thereof. In some embodiments, the UCDAI score for said patient is less than or equal to 1 after the 8 week period or after a treatment period of up to 8 weeks or a portion thereof. In other embodiments, the patient is experiencing an ulcerative colitis flare prior to said second composition being administered to said patient. In one embodiment, the second composition is administered to said patient once daily for an 8 week period or for up to 8 weeks or a portion thereof. In one embodiment, the ulcerative colitis in said patient is in remission after said 8 week period or after a treatment period of up to 8 weeks or a portion thereof.

Also provided are a first composition and a second composition for treating ulcerative colitis in a patient in need of such treatment, said patient having been previously administered a composition comprising 5-aminosalicylic acid. In one embodiment, the first composition is 5-aminosalicylic acid. In one embodiment, the second composition is in the form of a single tablet comprising about 9 mg of budesonide. In one embodiment, the second composition is administered to said patient for up to 8 weeks or a portion thereof. In some embodiments, the patient has a UCDAI score of greater than or equal to 4 prior to said second composition being administered to said patient. In another embodiment, the second composition is administered to said patient once daily for an 8 week period or for up to 8 weeks or a portion thereof. In some embodiments, the UCDAI score for said patient is less than or equal to 1 after the 8 week period or after a treatment period of up to 8 weeks or portion thereof. In other embodiments, the patient is experiencing an ulcerative colitis flare prior to said second composition being administered to said patient. In one embodiment, the second composition is administered to said patient once daily for an 8 week period or for up to 8 weeks or a portion thereof. In one embodiment, the ulcerative colitis in said patient is in remission after said 8 week period or after a treatment period of up to 8 weeks or a portion thereof. In some embodiments the first composition and second composition are administered to the patient simultaneously. In another embodiment, the amount of budesonide administered to the patient may be 9 mg per day and may be administered to the patient in a single dose or in a divided dose

Also provided are methods for maintaining remission of ulcerative colitis in a patient in need of such maintenance, said method comprising administering to said patient a composition in the form of a single tablet comprising about 6 mg of budesonide, whereby said remission of ulcerative colitis in said patient is maintained. In some embodiments, the single tablet comprising about 6 mg of budesonide is administered to said patient once daily for up to 12 months or a portion thereof. In other embodiments, the single tablet comprising about 6 mg of budesonide is administered to said patient once daily for up to 6 months or a portion thereof. In some embodiments, the composition in the form of a single tablet comprising 6 mg budesonide is the second composition described herein. In another embodiment, the amount of budesonide administered to the patient may be 6 mg per day and may be administered to the patient in a single dose or in a divided dose.

Also provided is a composition for maintaining remission of ulcerative colitis in a patient in need of such maintenance. In one embodiment, the composition is in the form of a single tablet comprising about 6 mg of budesonide, whereby said remission of ulcerative colitis in said patient is maintained. In some embodiments, the single tablet comprising about 6 mg of budesonide is administered to said patient once daily for up to 12 months or a portion thereof. In other embodiments, the single tablet comprising about 6 mg of budesonide is administered to said patient once daily for up to 6 months or a portion thereof. In some embodiments, the composition in the form of a single tablet comprising 6 mg budesonide is the second composition described herein. In another embodiment, the amount of budesonide administered to the patient may be 6 mg per day and may be administered to the patient

In some embodiments, the first composition comprises about 0.5 g to about 4.8 g 5-aminosalicylic acid and the second composition comprises about 1 mg to about 18 mg budesonide, such as about 0.8 g 5-aminosalicyclic acid and about 6 mg budesonide, or about 0.8 g 5-aminosalicyclic acid and about 9 mg budesonide, or about 2 g 5-aminosalicyclic acid and about 6 mg budesonide, or about 2 g 5-aminosalicyclic acid and about 9 mg budesonide, or about 2.4 g 5-aminosalicyclic acid and about 6 mg budesonide, or about 2.4 g 5-aminosalicyclic acid and about 9 mg budesonide, or about 4.2 g 5-aminosalicyclic acid and about 6 mg budesonide, or about 4.2 g 5-aminosalicyclic acid and about 9 mg budesonide.

In some embodiments, the first composition comprises about 400 mg to about 2000 mg rifamycin SV and the second composition comprises about 1 mg to about 18 mg budesonide, such as about 800 mg rifamycin SV and about 6 mg budesonide, or about 800 mg rifamycin SV and about 9 mg budesonide, or about 1200 mg rifamycin SV and about 6 mg budesonide, or about 1200 mg rifamycin SV and about 9 mg budesonide, or about 1800 mg rifamycin SV and about 6 mg budesonide, or about 1800 mg rifamycin SV and about 9 mg budesonide.

In some embodiments, wherein the first composition comprises about 500 mg to about 1500 mg ciprofloxacin and the second composition comprises about 1 mg to about 18 mg budesonide, such as about 500 mg ciprofloxacin and about 6 mg budesonide, or about 500 mg ciprofloxacin and about 9 mg budesonide, or about 750 mg ciprofloxacin and about 6 mg budesonide, or about 750 mg ciprofloxacin and about 9 mg budesonide, or about 1000 mg ciprofloxacin and about 6 mg budesonide, or about 1000 mg ciprofloxacin and about 9 mg budesonide.

### DEFINITIONS

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art.

The term “topically delivering” herein refers to the fact that certain compositions may be orally administered to a subject and the active ingredient(s) of the compositions may be released in such ways that the active ingredient(s) may reach the desired organ at a suitable time and extent so that it can exert its typical pharmacological activity, in our case represented by some areas of the gastrointestinal tract. In one embodiment, the active ingredient is budesonide. In one embodiment, the composition comprises about 9 mg budesonide. In one embodiment, the composition comprises about 6 mg budesonide. In one embodiment, the composition comprises about 4.5 mg budesonide. In one embodiment, the composition comprises about 3 mg budesonide. In one embodiment, the composition comprises about 12 mg budesonide. In one embodiment, the composition comprises about 15 mg budesonide. In one embodiment, the composition comprises about 18 mg budesonide.

The terms “5-ASA” and “mesalamine” used herein refer to 5-aminosalicylic acid, or a pharmaceutically acceptable salt thereof.

The term “remission” used herein generally refers to a reduction in the symptoms of a patient suffering from IBD, such as ulcerative colitis and Crohn's Disease. In some instances, remission may be measured as reduction in an individual patient's UCDAI score, which is well known and can be measured using methods known to those of ordinary skill in the art. UCDAI is a four-component scale (total score of 0 to 12) that encompasses the clinical assessments of stool frequency, rectal bleeding, mucosal appearance and physician's rating of disease activity (score of 0 to 3 for each of the components). In some embodiments, remission is defined as the reduction in a patient's UCDAI score from greater than 1 to a UCDAI score of ≦1, with subscores of 0 for rectal bleeding, stool frequency, and mucosal appearance and with a ≧1 point reduction in an endoscopy-only score. In other instances, remission may be measured as reduction in Clinical Activity Index (CAI), which is well known and can be measured using methods known to those of ordinary skill in the art. In some embodiments, remission is defined in a patient's CAI score from greater than 4 to a CAI score of no more than 4.

The term “intestinal disease presenting at least one inflammatory component” used herein refers to diseases or conditions known to those of ordinary skill in the art as intestinal diseases which includes inflammation. Examples of inflammatory components are reddening, swelling, exudation, friability, hemorrhage, ulceration. The intestinal disease may be chronic or, in some instances, acute. An example of a chronic intestinal disease presenting at least one inflammatory component is inflammatory bowel disease. An example of an acute intestinal disease presenting at least one inflammatory component is acute diverticulitis.

The terms “IBD” and “inflammatory bowel disease” used herein refer to conditions known to those of ordinary skill in the art as inflammatory bowel disease, such as ulcerative colitis and Crohn's Disease. In some embodiments, the inflammatory bowel disease is ulcerative colitis. In some embodiments, the ulcerative colitis may be active mild to medium ulcerative colitis. In some embodiments, the inflammatory bowel disease is Crohn's Disease.

In any of the embodiments described herein that describe the treatment of and/or maintaining the remission of a chronic intestinal disease are understood to refer to the treatment and/or maintaining the remission of inflammatory bowel disease including ulcerative colitis, active mild to medium ulcerative colitis and Crohn's disease In any of the embodiments described herein that describe the treatment of and/or maintaining the remission of an acute intestinal disease are understood to refer to the treatment and/or maintaining the remission of acute diverticulitis.

The term “UC” used herein refers to ulcerative colitis.

The term “matrix” used herein refers to a macroscopically homogeneous structure in all its volume.

The term “matrix-forming excipient” used herein refers to an excipient having chemical-physical characteristics, such as lipophilia, amphiphilia, hydrophilia, or swelling properties as regards hydrophilic matrix, that one of ordinary skill in the art would recognize provide useful properties in preparing and using the compositions described herein. One or more matrix forming-excipients may comprise a matrix. Thus a matrix may comprise one matrix-forming excipient or more than one matrix-forming excipients. For example, a lipophilic matrix can comprise one fatty acid, or it may comprise one or more fatty acids or a fatty acid and a wax. Optionally, other physiologically acceptable excipients (e.g. active substance or lubricants) can be added to the matrix-forming excipient or to a mixture of these compounds to form and confer the desired characteristics to the compositions described herein, such as a lipophilic matrix, and/or an amphiphilic matrix and/or a hydrophilic matrix. One of ordinary skill in the art will recognize that an excipient comprising the compositions described herein may possess more than one characteristic that is useful in preparing and using the compositions disclosed herein. For example, an excipient, such as magnesium stearate or stearic acid, may function as a matrix-forming material and as a lubricant. Furthermore, one of ordinary skill in the art will recognize that there is no particular amount of an excipient that must be present in the compositions disclosed herein in order for such excipient to function as a matrix-forming excipient.

The term “simultaneous, separate or sequential use” used herein refers to administration of the first and second compositions at the same time or in such a manner that the two compositions act in the patient's body at the same time or administration of one composition after the other composition in such a manner to provide a therapeutic effect. In some embodiments the compositions are taken with a meal. In other embodiments, the compositions are taken after a meal, such as 30 minutes or 60 minutes after a meal. In some embodiments, one composition is administered to a patient for a time period followed by administration of the other composition.

### DETAILED DESCRIPTION OF THE DISCLOSURE

Provided herein are methods for treating inflammatory bowel disease and/or maintaining remission of inflammatory bowel disease in a patient previously administered a first composition for treatment of said disease, comprising administering to said patient a second composition comprising: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein is a second composition for treating inflammatory bowel disease and/or maintaining remission of inflammatory bowel disease in a patient previously treated with a first composition for treatment of said disease. In one embodiment, the second composition comprises: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein are methods for treating inflammatory bowel disease and/or maintaining remission of inflammatory bowel disease in a patient simultaneously administered a first composition for treatment of said disease comprising: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) a coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein is a second composition for treating inflammatory bowel disease and/or maintaining remission of inflammatory bowel disease in a patient simultaneously treated with a first composition for treatment of said disease. In one embodiment, the second composition comprises: (1) a tablet core comprising: (a) budesonide in an amount effective for treating inflammatory bowel disease and/or maintaining remission of inflammatory bowel disease, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein are methods for treating intestinal disease presenting at least one inflammatory component and/or maintaining remission of intestinal disease presenting at least one inflammatory component in a patient previously administered a first composition for treatment of said disease, comprising administering to said patient a second composition comprising: (1) a tablet core comprising: (a) budesonide in an amount effective for treating intestinal disease presenting at least one inflammatory component and/or maintaining remission of intestinal disease presenting at least one inflammatory component, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided herein is a second composition for treating intestinal disease presenting at least one inflammatory component and/or maintaining remission of intestinal disease presenting at least one inflammatory component in a patient previously treated with a first composition for treatment of said disease. In one embodiment, the second composition comprises: (1) a tablet core comprising: (a) budesonide in an amount effective for treating intestinal disease presenting at least one inflammatory component and/or maintaining remission of intestinal disease presenting at least one inflammatory component, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least hydrophilic excipient is a hydrophilic matrix-forming excipient.

Also provided herein are methods for treating intestinal disease presenting at least one inflammatory component and/or maintaining remission of intestinal disease presenting at least one inflammatory component in a patient simultaneously administered a first composition for treatment of said disease, comprising administering to said patient a second composition comprising: (1) a tablet core comprising: (a) budesonide in an amount effective for treating intestinal disease presenting at least one inflammatory component and/or maintaining remission of intestinal disease presenting at least one inflammatory component, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least hydrophilic excipient is a hydrophilic matrix-forming excipient.

Also provided herein is a second composition for treating intestinal disease presenting at least one inflammatory component and/or maintaining remission of intestinal disease presenting at least one inflammatory component in a patient simultaneously treated with a first composition for treatment of said disease. In one embodiment, the second composition comprises: (1) a tablet core comprising: (a) budesonide in an amount effective for treating intestinal disease presenting at least one inflammatory component and/or maintaining remission of intestinal disease presenting at least one inflammatory component, (b) at least one lipophilic excipient; (c) at least one amphiphilic excipient; and (d) at least one hydrophilic excipient; and (2) at least one coating on said tablet core, said coating comprising a gastro-resistant film. In some embodiments, the tablet core is a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

Also provided are methods for treating ulcerative colitis in a patient in need of such treatment, said patient having been previously administered a composition comprising 5-aminosalicylic acid, said method comprising administering to said patient a second composition in the form of a single tablet comprising about 9 mg of budesonide.

Also provided is a second composition for treating ulcerative colitis in a patient in need of such treatment, said patient having been previously treated with a composition comprising 5-aminosalicylic acid. In one embodiment, the second composition is in the form of a single tablet comprising about 9 mg of budesonide.

Also provided are methods for treating ulcerative colitis in a patient in need of such treatment, said method comprising administering to said patient a first and a second composition, said first composition comprising 5-aminosalicylic acid and said second composition is in the form of a single tablet comprising about 9 mg of budesonide.

Also provided are a first composition and a second composition for treating ulcerative colitis in a patient in need of such treatment. In one embodiment, the first composition comprises 5-aminosalicylic acid.

Also provided are methods for treating ulcerative colitis in a patient in need of such treatment, said patient having been previously administered a composition comprising 5-aminosalicylic acid, said method comprising administering to said patient a first composition and a second composition, said first composition comprising 5-aminosalicylic acid and said second composition is in the form of a single tablet comprising about 9 mg of budesonide.

Also provided are a first composition and a second composition for treating ulcerative colitis in a patient in need of such treatment, said patient having been previously administered a composition comprising 5-aminosalicylic acid. In one embodiment, the first composition comprises 5-aminosalicylic acid. In one embodiment, the second composition is in the form of a single tablet comprising about 9 mg of budesonide.

Also provided are methods for maintaining remission of ulcerative colitis in a patient in need of such maintenance, said method comprising administering to said patient a composition in the form of a single tablet comprising about 6 mg of budesonide, whereby said remission of ulcerative colitis in said patient is maintained. In some embodiments, the composition in the form of a single tablet comprising 6 mg budesonide is the second composition described herein.

Also provided is a composition for maintaining remission of ulcerative colitis in a patient in need of such maintenance. In one embodiment, the composition is in the form of a single tablet comprising about 6 mg of budesonide, whereby said remission of ulcerative colitis in said patient is maintained. In some embodiments, the composition in the form of a single tablet comprising 6 mg budesonide is the second composition described herein.

Also provided is a medicament comprising in combination a first and a second composition for simultaneous, separate or sequential use in treating an intestinal disease presenting at least one inflammatory component and/or maintaining remission of an intestinal disease presenting at least one inflammatory component in a patient, said first composition comprising at least a compound for treating said disease, said second composition comprising:

(1) a tablet core comprising:
a) budesonide in an amount effective for treating or maintaining remission of an intestinal disease presenting at least one inflammatory component,
b) at least one lipophilic excipient;
c) at least one amphiphilic excipient; and
d) at least one hydrophilic excipient; and

(2) a coating on said tablet core, said coating comprising a gastro-resistant film.

First Composition

The first composition may comprise at least one compound chosen from systemic corticosteroids and non-systemic corticosteroids. In some embodiments, first composition comprises a dose of a compound for treating inflammatory bowel disease (IBD), such as, ulcerative colitis or Crohn's Disease. The compound may be a small molecule compound or a biologic such as an antibody, a protein or peptide, or an RNA- or DNA-based drug.

In some embodiments, the first composition may be a dose of an oral composition comprising 5-ASA, also known as mesalamine, (e.g., ASACOL®, ASACOL® HD, LIALDA®, PENTASA®, and DELZICOL™), or a mesalamine prodrug, for example, sulfasalazine (e.g., AZULFIDINE®), olsalazine (e.g., DIPENTUM®), and balsalazide (COLAZAL®, COLAZIDE®). For mesalamine, a daily dose may be, for example, at least 0.5 g, or at least 0.8 g, or at least 1.6 g, or at least 2 g, or at least 2.4 g, or at least 3 g, or at least 3.2 g, or at least 3.8 g, or at least 4.2 g, or at least 4.6 g, or at least 4.8 g. In some embodiments, the first composition comprises at least 2.4 g mesalamine. In other embodiments, the first composition comprises about 2.4 g mesalamine. For sulfasalazine, a dose may be, for example, at least 2 g, or at least 4 g. In some embodiments, the first composition comprises at least 4 g sulfasalazine. For olsalazine, a dose may be, for example, at least 1 g, or at least 2 g. In some embodiments, the first composition comprises at least 2 g olsalazine. For balsalazide, a dose may be, for example, at least 3 g, at least 6 g. In some embodiments, the first composition comprises at least 6.75 g balsalazide.

In some embodiments, the first composition comprises a dose of an oral budesonide. For example, the first composition may comprise budesonide in an amount ranging from 0.1 mg to 20 mg, for example, 5-15 mg, or 5-10 mg, or any amount within the range of 0.1-20 mg, for example 1-12 mg or 1-18 mg. In some embodiments, the first composition comprises about 6 mg budesonide. In some embodiments, the first composition comprises about 4.5 mg budesonide. In some embodiments, the first composition comprises about 3 mg budesonide. In some embodiments, the first composition comprises about 9 mg budesonide. In some embodiments, the fast composition comprises about 18 mg budesonide. In some embodiments, the first composition comprises about 15 mg budesonide. In some embodiments, the first composition comprises about 12 mg budesonide.

In an add-on therapy in which the patient has been previously treated for IBD with a first composition, the patient may have been treated with a first composition for a period of at least 1 week, at least 2 weeks, at least 4 weeks, at least 8 weeks, at least 16 weeks, or at least 1 year, or any time period desirable for inducing or maintaining remission of IBD.

In an add-on therapy in which the patient has been previously treated for an intestinal disease presenting at least one inflammatory component with a first composition, the patient may have been treated with a first composition for a period of at least 1 week, at least 2 weeks, at least 4 weeks, at least 8 weeks, at least 16 weeks, or at least 1 year, or any time period desirable for inducing or maintaining remission of an intestinal disease presenting at least one inflammatory component.

In some embodiments, the first composition comprises at least one compound selected from systemic or topical antibiotics or sulphonamides or antinfective chemotherapeutics or antinfective compounds or motility-controlling drugs or their combinations. In other embodiments, the first composition comprises absorbable antibiotics or unabsorbable antibiobiotics, betalactamic antibiotics, chinolones, and/or their combionation with other substances. In some embodiments, the first composition comprises a stable dose of ampicillin or amoxicillin or ciprofloxacin or fidaxomicin or erythromycin or paromomycine or trimethoprim-sulphamethoxazole or metronidazole or vancomycin or Bismuth salts and derivatives or rifaximine or rifamycin SV or chloramphenicol or streptomycin or bacitracin or neomycin or their combinations. In some embodiments, the first composition comprises rifamycin SV in an amount from about 400 mg to about 2000 mg, such as about 800 mg or about 1200 mg or about 1800 mg. In other embodiments, the second composition comprises ciprofloxacin in an amount from about 500 mg to about 1500 mg, such as about 500 mg or about 750 mg or about 1000 mg.

Second Composition

The second composition may comprise a tablet comprising budesonide. In one embodiment, the tablet may comprise budesonide in an amount effective for treating inflammatory bowel disease and/or maintaining remission of inflammatory bowel disease, such as 3 mg budesonide, 4.5 mg budesonide, 6 mg budesonide, or 9 mg of budesonide, or 12 mg budesonide, or 15 mg budesonide, or 18 mg budesonide. In another embodiment, the tablet may comprise budesonide, at least one lipophilic excipient, at least one amphiphilic excipient, and at least one hydrophilic excipient.

The second composition may comprise a tablet core and a coating on the tablet core. The tablet core may comprise budesonide in an amount effective for treating inflammatory bowel disease and/or maintaining remission of inflammatory bowel disease, at least one lipophilic excipient, at least one amphiphilic excipient, and at least one hydrophilic excipient.

The second composition may comprise a tablet comprising budesonide. In one embodiment, the tablet may comprise budesonide in an amount effective for an intestinal disease presenting at least one inflammatory component and/or maintaining remission of an intestinal disease presenting at least one inflammatory component, such as 1-12 mg budesonide, 1-18 budesonide, 3 mg budesonide, 4.5 mg budesonide, 6 mg budesonide, 9 mg of budesonide, 12 mg budesonide, 15 mg budesonide or 18 mg budesonide. In another embodiment, the tablet may comprise budesonide, at least one lipophilic excipient, at least one amphiphilic excipient, and at least one hydrophilic excipient. In some embodiments, the tablet may be a multi-matrix tablet. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

In some embodiments, the second composition comprises about 1 mg to 18 mg budesonide as a single dose or as a divided dose. In one embodiment, the second composition comprises about 1 mg to 12 mg budesonide as a single dose or as a divided dose. In another embodiment, the second composition comprises about 18 mg budesonide as a single dose or as a divided dose. In another embodiment, the second composition comprises about 15 mg budesonide as a single dose or as a divided dose. In another embodiment, the second composition comprises about 12 mg budesonide as a single dose or as a divided dose. In another embodiment, the second composition comprises about 9 mg budesonide as a single dose or as a divided dose. In another embodiment, the second composition comprises about 6 mg budesonide as a single dose or as a divided dose. In another embodiment, the second composition comprises about 4.5 mg budesonide as a single dose or as a divided dose. In another embodiment, the second composition comprises about 3 mg budesonide as a single dose or as a divided dose.

The second composition may comprise a tablet core and a coating on the tablet core. The tablet core may comprise budesonide in an amount effective for treating an intestinal disease presenting at least one inflammatory component and/or maintaining remission of an intestinal disease presenting at least one inflammatory component, at least one lipophilic excipient, at least one amphiphilic excipient, and at least one hydrophilic excipient. In some embodiments, the tablet core may be a multi-matrix tablet core. In one embodiment, at least one lipophilic excipient is a lipophilic matrix-forming excipient. In another embodiment, at least one amphiphilic excipient is an amphiphilic matrix-forming excipient. In another embodiment, at least one hydrophilic excipient is a hydrophilic matrix-forming excipient.

In some embodiments, the tablet core may comprise budesonide in an amount ranging from 0.1 mg to 20 mg, for example, 3-15 mg, or 5-10 mg, or any amount within the range of 0.1-20 mg, for example, 1-12 mg or 1-18 mg. In some embodiments, the tablet core comprises about 3 mg budesonide. In some embodiments, the tablet core comprises about 6 mg budesonide. In some embodiment, the tablet core comprises about 9 mg budesonide. In some embodiments, the tablet core comprises about 4.5 mg budesonide. In some embodiments, the tablet core comprises about 3 mg budesonide. In some embodiments, the tablet core comprises about 12 mg budesonide. In some embodiments, the tablet core comprises about 15 mg budesonide. In some embodiments, the tablet core comprises about 18 mg budesonide.

In some embodiments, the at least one lipophilic excipient or the at least one lipophilic matrix-forming excipient may be chosen from unsaturated or hydrogenated alcohols or fatty acids, salts, esters or amides thereof, fatty acids mono-, di- or triglycerides, the polyethoxylated derivatives thereof, waxes, ceramides, cholesterol derivatives or mixtures thereof. In some embodiments, the at least one lipophilic excipient or the at least one lipophilic matrix-forming excipient may have a melting point within the range of 40 to 90° C., for example, from 50 to 80° C., or from 60 to 70° C. In some embodiments, the at least one lipophilic excipient or the at least one lipophilic matrix-forming excipient may be selected from saturated or unsaturated fatty acids, such as palmitic, stearic, myristic, lauric, laurylic, or oleic acids or mixtures thereof with other fatty acids with shorter chain. In some embodiments, the at least one lipophilic excipient or the at least one lipophilic matrix-forming excipient may be stearic acid. If desired, a fatty acid calcium salt may be incorporated in the at least one lipophilic excipient or in the at least one lipophilic matrix-forming excipient. In some embodiments and if desired, a physiologically acceptable salt of a fatty acid, such as sodium, calcium or magnesium salts, may be incorporated in the at least one lipophilic excipient or in the at least one lipophilic matrix-forming excipient.

In some embodiments, the at least one amphiphilic excipient or the at least one amphiphilic matrix-forming excipient may be chosen from polar lipids of type I or II (lecithin, phosphatidylcholine, phosphatidylethanolamine), ceramides, glycol alkyl ethers such as diethylene glycol monomethyl ether (e.g. TRANSCUTOL®). In some embodiments, the at least one amphiphilic excipient or the at least one amphiphilic matrix-forming excipient may be lecithin.

In some embodiments, the at least one hydrophilic excipient or the at least one hydrophilic matrix-forming excipient may be chosen from hydrogels, i.e. substances which when passing from the dry state to the hydrated one, undergo the so-called “macromolecular relaxation,” namely a remarkable increase in mass and weight following the coordination of a large number of water molecules by the polar groups present in the polymeric chains of the excipients themselves. Examples of hydrogels include, but are not limited to, acrylic or methacrylic acid polymers or copolymers, alkylvinyl polymers, hydroxyalkyl celluloses, carboxyalkyl celluloses, polysaccharides, dextrins, pectins, starches and derivatives, natural or synthetic gums, and alginic acid. In some embodiments, the at least one hydrophilic excipient or the at least one hydrophilic matrix-forming excipient may be chosen from polyalcohols such as xylitol, maltitol and mannitol. In some embodiments, the at least one hydrophilic excipient or the at least one hydrophilic matrix-forming excipient may be chosen from hydroxyalkyl celluloses. In some embodiments, the at least one hydrophilic excipient or the at least one hydrophilic matrix-forming excipient may be hydroxypropylcellulose, hydroxypropylmethylcellulose, an alkylcellulose derivative, or a hydroxyalkylcellulose derivative or a mixture thereof. In some embodiments, the hydrophilic excipient or the hydrophilic matrix-forming excipient may be hydroxypropylcellulose, hydroxypropylmethylcellulose, or a mixture thereof. In some embodiments, the hydrophilic excipient or the hydrophilic matrix-forming excipient may be hydroxypropylcellulose. In some embodiments, the hydrophilic excipient or the hydrophilic matrix-forming excipient may be hydroxypropylmethylcellulose. In some embodiments, the hydrophilic excipient is not a natural or synthetic gum. In some embodiments, the hydrophilic excipient is not a natural gum. In some embodiments, the hydrophilic excipient is not a synthetic gum. In some embodiments, the hydrophilic excipient is not guar gum.

The coating on said tablet core may comprise a gastro-resistant film. The gastro-resistant film may be chosen from acrylic and/or methacrylic acids polymers or copolymers (EUDRAGIT® R or S/L) or cellulose derivatives, such as cellulose acetophthalate/s. In some embodiments, the gastro-resistant film may be chosen from at least one methacrylic acid polymer or copolymer or a mixture of methacrylic acid polymers or copolymers. In some embodiments, the gastro-resistant film may be a mixture of acrylic and/or methacrylic acid copolymers type A and/or type B (for example, EUDRAGIT® S100 and/or EUDRAGIT® L100 or EUDRAGIT® RS). In some embodiments, the gastro-resistant film may be methacrylic acid copolymer types A and B.

In some embodiments, the second composition may further comprise at least one other pharmaceutically acceptable excipient. In some embodiments, the at least other one pharmaceutically acceptable excipient may be chosen from microcrystalline cellulose, lactose, silicon dioxide (silica), magnesium stearate, talc, triethylcitrate, and titanium dioxide.

In some embodiments, the second composition may comprise about 9 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In some embodiments, the second composition may comprise about 6 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In some embodiments, the second composition may comprise about 4.5 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In some embodiments, the second composition may comprise about 3 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In some embodiments, the second composition may comprise about 12 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In some embodiments, the second composition may comprise about 15 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

In some embodiments, the second composition may comprise about 18 mg budesonide, stearic acid, lecithin, microcrystalline cellulose, hydroxypropylcellulose, lactose, silicon dioxide, magnesium stearate, methacrylic acid copolymer types A and B, talc, triethylcitrate, and titanium dioxide.

Non-limiting exemplary embodiments of the second composition and non-limiting exemplary embodiments for preparing the second composition may be found in U.S. Pat. Nos. 7,431,943, 7,410,651, RE43,799, 8,293,273, and U.S. Patent Application Publication No. 2012/0021052, all of which are hereby incorporated by reference in their entireties.

The second composition may be administered to a patient who is in need of treatment of mild to moderate IBD. In some embodiments, the patient may be in need of maintaining remission of IBD. In some embodiments, the IBD is ulcerative colitis or Crohn's Disease. In some embodiments, the IBD is mild to moderate active ulcerative colitis (also referred to as active mild to moderate ulcerative colitis). In some embodiments, the second composition comprises budesonide and is administered to patients for the induction of remission in patients with active, mild to moderate ulcerative colitis. In another embodiment, the second composition comprises budesonide and is administered to patients for the induction of remission in patients with active, mild to moderate ulcerative colitis with an inadequate response to the administration of a first composition comprising mesalamine. In one embodiment, the second composition comprises about 9 mg budesonide. In another embodiment, the second composition comprises about 6 mg budesonide. In another embodiment, the second composition comprises about 4.5 mg budesonide. In another embodiment, the second composition comprises about 3 mg budesonide.

The second composition may be administered to a patient who is in need of treatment of an intestinal disease presenting at least one inflammatory component. In some embodiments, the patient may be in need of maintaining remission of intestinal disease presenting at least one inflammatory component. In some embodiments, intestinal disease presenting at least one inflammatory component is a chronic disease or an acute or acute phase of a disease. In one embodiment the chronic disease is IBD. In one embodiment, the IBD is ulcerative colitis or Crohn's Disease. In some embodiments, the IBD is active mild to moderate active ulcerative colitis (also referred to as active mild to moderate ulcerative colitis). In one embodiment, the disease is an acute disease or an acute phase of a disease. In some embodiments, the acute disease is acute diverticulitis. In some embodiments, the second composition comprises budesonide and is administered to patients for the induction of remission in patients with active, mild to moderate ulcerative colitis. In another embodiment, the second composition comprises budesonide and is administered to patients for the induction of remission in patients with active, mild to moderate ulcerative colitis with an inadequate response to the administration of a first composition comprising mesalamine. In another embodiment, the second composition comprises budesonide and is administered to patients for the induction of remission of the acute phase of diverticular disease. In one embodiment, the second composition comprises about 18 mg budesonide. In one embodiment, the second composition comprises about 15 mg budesonide. In one embodiment, the second composition comprises about 12 mg budesonide. In one embodiment, the second composition comprises about 9 mg budesonide. In another embodiment, the second composition comprises about 6 mg budesonide. In another embodiment, the second composition comprises about 4.5 mg budesonide. In another embodiment, the second composition comprises about 3 mg budesonide.

In some embodiments, the second composition may be administered for a time period of at least 1 week, at least 2 weeks, at least 4 weeks, at least 8 weeks, at least 16 weeks, at least 6 months, at least 9 months, at least 12 months, or at least 24 months, or any time period desirable for inducing or maintain remission of IBD. In one embodiment, the IBD is ulcerative colitis. In another embodiment, the IBD is Crohn's Disease.

In some embodiments, the second composition may be administered for a time period of at least 1 week, at least 2 weeks, at least 4 weeks, at least 8 weeks, at least 16 weeks, at least 6 months, at least 9 months, at least 12 months, or at least 24 months, or any time period desirable for inducing or maintain remission of an intestinal disease presenting at least one inflammatory component.

In another embodiment, the second composition comprises 9 mg of budesonide and is administered to a patient once daily in the morning with or without food for up to 8 weeks. In another embodiment, the second composition comprises a delayed and extended release tablet comprising about 9 mg of budesonide and is administered to a patient once daily in the morning with or without food for up to 8 weeks. In some embodiments, the amount of budesonide in the second composition may be any amount described herein, for example, 1-12 mg, 1-18 mg, 3 mg, 4.5 mg, 6 mg, 9 mg, 12 mg, 15 mg or 18 mg.

In some embodiments, the second composition comprises tablets that should be swallowed whole with water and not chewed, crushed or broken. In another embodiment, the patients to whom the second composition is administered should be advised to avoid the consumption of grapefruit juice for the duration of the time during which they are being administered the second composition.

In some embodiments, the first composition comprises about 0.5 g to about 4.8 g 5-aminosalicylic acid and the second composition comprises about 1 mg to about 18 mg budesonide, such as about 0.8 g 5-aminosalicyclic acid and about 6 mg budesonide, or about 0.8 g 5-aminosalicyclic acid and about 9 mg budesonide, or about 2 g 5-aminosalicyclic acid and about 6 mg budesonide, or about 2 g 5-aminosalicyclic acid and about 9 mg budesonide, or about 2.4 g 5-aminosalicyclic acid and about 6 mg budesonide, or about 2.4 g 5-aminosalicyclic acid and about 9 mg budesonide, or about 4.2 g 5-aminosalicyclic acid and about 6 mg budesonide, or about 4.2 g 5-aminosalicyclic acid and about 9 mg budesonide.

In some embodiments, the first composition comprises about 400 mg to about 2000 mg rifamycin SV and the second composition comprises about 1 mg to about 18 mg budesonide, such as about 800 mg rifamycin SV and about 6 mg budesonide, or about 800 mg rifamycin SV and about 9 mg budesonide, or about 1200 mg rifamycin SV and about 6 mg budesonide, or about 1200 mg rifamycin SV and about 9 mg budesonide, or about 1800 mg rifamycin SV and about 6 mg budesonide, or about 1800 mg rifamycin SV and about 9 mg budesonide.

In some embodiments, wherein the first composition comprises about 500 mg to about 1500 mg ciprofloxacin and the second composition comprises about 1 mg to about 18 mg budesonide, such as about 500 mg ciprofloxacin and about 6 mg budesonide, or about 500 mg ciprofloxacin and about 9 mg budesonide, or about 750 mg ciprofloxacin and about 6 mg budesonide, or about 750 mg ciprofloxacin and about 9 mg budesonide, or about 1000 mg ciprofloxacin and about 6 mg budesonide, or about 1000 mg ciprofloxacin and about 9 mg budesonide.

### EXAMPLES

The embodiments described below may be modified using methods known to those of ordinary skill in the art to prepare dosage forms, such as tablets, each of which contains from about 1 mg to about 20 mg budesonide, such as about 1 mg to about 12 mg budesonide, about 1 mg to about 18 mg, budesonide, about 18 mg budesonide, about 15 mg budesonide, about 12 mg budesonide, about 9 mg budesonide, about 6 mg budesonide, about 4.5 mg budesonide, or about 3 mg budesonide.

Unless otherwise indicated, the dissolution test described herein is performed by introducing individual tablets in a vessel containing from 500 to 1000 ml of a buffered solution set to pH 7.2, so that the pH conditions of large intestine should be reproduced. To simulate the human body conditions, the test is carried out at a temperature of 37° C.±2° C. and at predetermined time periods samples of the dissolution medium are withdrawn to detect the percentage of active ingredient dissolved over time.

### Example 1

### Budesonide MMX® Composition

2.7 kg of budesonide, 3.0 kg of lecithin (amphiphilic excipient) and 3.0 kg of stearic acid (lipophilic excipient) are mixed after sieving till a homogeneous mixture is obtained. Then 39.0 kg of inert, functional excipients and 9.0 kg of low viscosity hydroxypropylcellulose (binder) are added and mixed for 10 minutes before adding purified water and kneading to a suitable consistency. Then pass the granulate through a rotating granulator equipped with the suitable screen and transfer the granulate to the fluid bed drier to lower the residual moisture content under 3%. After a new sieving of the dried granulate, 9.0 kg of hydroxypropylcellulose (hydrophilic excipient) and the suitable amount of functional excipients (in particular, microcrystalline cellulose, lactose and silicon dioxide) are added, and, after 15 minutes of mixing, magnesium stearate in a suitable quantity to act as lubricant is added.

After a final blending, tablets of around 300 mg of unitary weight are generated.

The core are then coated with a suspension obtained introducing into a stainless steel container 5.8 kg of Eudragit™ (methacrylate copolymers), 0.6 kg of triethylcitrate and 3.0 kg of dyes and talc, using alcohol as solvent.

The mean dissolution percentage (as average of six or more tablets) obtained with this tablet formulation were around 10-20% at second hour sampling, in the range 25% to 65% at fourth hour and a dissolution greater than 80% was achieved at 8^{th} hour sampling.

### Example 2

| | |
|---|---|
| Component | mg/tablet |

Tablet

| | |
|---|---|
| Budesonide | 9.0 |
| Stearic Acid (lipophilic matrix forming materials) | 10.0 |
| Lecithin (amphiphilic matrix forming material) | 10.0 |
| Microcrystalline cellulose | 156.0 |
| Hydroxypropylcellulose | 60.0 |
| Lactose monohydrate | 50.0 |
| Silica, colloidal hydrated | 2.0 |
| Magnesium stearate | 3.0 |

Coating materials

| | |
|---|---|
| Eudragit L100 (acrylic and methacrylic copolymer) | 14.0 |
| Eudragit S100 (acrylic and methacrylic copolymer) | 12.0 |
| Talc | 7.9 |
| Titanium dioxide | 4.5 |
| Triethylcitrate | 1.6 |
| Alcohol | q.s. |

The coating of industrial scale tablets of batch MV084 contained 8.0 mg of Eudragit L100 and 8.0 mg of Eudragit S100 (instead of 14.0 mg and 12.0 mg, respectively) with an individual weight of about 330 mg.

According to the present disclosure, coated tablets individually weighing about 330 mg or about 340 mg are obtained.

The above described dissolution test is performed on the tablets of Example 2. The results are the following (indicated as average value):

| after 2 hours at pH 1 | resistent (<5%) |
|---|---|
| after 1 hour at pH 6.4 | resistent (<5%) |
| after 2 hours at pH 7.2 | 15% |
| after 4 hours at pH 7.2 | 37% |
| after 8 hours at pH 7.2 | 91% |

### Example 2A

### Budesonide MMX® Composition

Tablets comprising 9 mg of budesonide and having the following composition were prepared having an individual weight of about 330 mg.

| | |
|---|---|
| Component | mg/tablet |

Tablet

| | |
|---|---|
| Budesonide | 9.0 |
| Stearic Acid (lipophilic matrix forming materials) | 10.0 |
| Lecithin (amphiphilic matrix forming material) | 10.0 |
| Microcrystalline cellulose | 156.0 |
| Hydroxypropylcellulose | 60.0 |
| Lactose monohydrate | 50.0 |
| Silica, colloidal hydrated | 2.0 |
| Magnesium stearate | 3.0 |

Coating materials

| | |
|---|---|
| Eudragit L100 (methacrylic copolymer, Type A) | 8.0 |
| Eudragit S100 (methacrylic copolymer, Type B) | 8.0 |
| Talc | 7.9 |
| Titanium dioxide | 4.5 |
| Triethylcitrate | 1.6 |
| Alcohol | q.s. |

Coated tablets individually weighing about 330 mg were obtained.

### Example 2B

### Budesonide MMX® Composition

Tablets comprising 6 mg of budesonide and having the following composition were prepared having an individual weight of about 330 mg.

| | |
|---|---|
| Component | mg/tablet |

Tablet

| | |
|---|---|
| Budesonide | 6.0 |
| Stearic Acid (lipophilic matrix forming materials) | 10.0 |
| Lecithin (amphiphilic matrix forming material) | 10.0 |
| Microcrystalline cellulose | 156.0 |
| Hydroxypropylcellulose | 60.0 |
| Lactose monohydrate | 53.0 |
| Silicon dioxide | 2.0 |
| Magnesium stearate | 3.0 |

Coating materials

| | |
|---|---|
| Eudragit L100 (acrylic and methacrylic copolymer) | 8.0 |
| Eudragit S100 (acrylic and methacrylic copolymer) | 8.0 |
| Talc | 7.9 |
| Titanium dioxide | 4.5 |
| Triethylcitrate | 1.6 |
| Alcohol | q.s. |

Coated tablets individually weighing about 330 mg were obtained.

### Example 3

### Budesonide MMX® Composition

Budesonide (3.0 kg) is mixed with soybean Lecithin (5.0 kg) until a homogeneous mixture is obtained. Then carnauba wax (2.0 kg) and stearic acid (2.0 kg) sieved through a fine screen are added. After mixing, the powders are added with other functional excipients and kneaded with a binder solution obtained by dissolving medium viscosity polyvinylpirrolidone in water. After drying in a fluid bed and milling throughout a suitable screen, hydroxypropylmethylcellulose (35.0 kg) and other excipients, including magnesium stearate as lubricant, in a suitable quantity are added and the mixture is blended till a homogeneous powder dispersion is obtained.

The powder mixture is subjected to compression in a rotating tabletting machine and the tablets so obtained are coated in a pan coat with a gastroresistant composition containing Eudragit™, plasticizers, dyes and pigments.

According to the present example, coated tablets individually weighing around 105 mg are obtained.

The results of the above described dissolution test are the following (indicated as average value of at least six tablets):

| after 2 hours at pH 1 | resistant (<5%) |
|---|---|
| after 1 hour at pH 6.4 | resistant (<5%) |
| after 2 hours at pH 7.2 | 9% |
| after 4 hours at pH 7.2 | 28% |
| after 8 hours at pH 7.2 | 86% |

### Example 4

### Budesonide MMX® Composition

50 g of diethylene glycol monoethyl ether are homogeneously distributed on 500 g of microcrystalline cellulose; then 100 g of budesonide are added, mixing to complete homogenization. This mix is further added with 400 g of budesonide, then dispersed in a blender containing 100 g of carnauba wax and 100 g of stearic acid preheated at a temperature of 60° C. After kneading for 5 minutes, the mixture is cooled to room temperature and extruded in granules of size below 1 mm. A suitable mixer is loaded with the matrix granules prepared as above and the following amounts of hydrophilic excipients: 1500 g of hydroxypropyl methylcellulose and 500 g of Policarbophil™ are added. The components are mixed until homogeneous dispersion of the matrices, then added with 2450 g of microcrystalline cellulose, 400 g of lactose, 100 g of colloidal silica and 50 g of magnesium stearate. After further 5 minute mixing, the mix is tableted to unitary weight of 250 mg/tablet.

Tablets are then subjected to coating using a suspension containing polyacrylate and polymethacrilate copolymers in addition to dyes, plasticizers and colouring agents in solvent (ethyl alcohol).

The results of the dissolution test performed on these coated tablets are the following (indicated as average value of at least six tablets):

| after 2 hours at pH 1 | resistant (<5%) |
|---|---|
| after 1 hour at pH 6.4 | resistant (<5%) |
| after 2 hours at pH 7.2 | 11% |
| after 4 hours at pH 7.2 | 32% |
| after 8 hours at pH 7.2 | 76% |

### Example 5

### Budesonide MMX® for the Maintenance of Remission in Patients with Ulcerative Colitis (UC)

Budesonide MMX (budesonide MMX) 9 mg tablets having the composition as in Example 2A administered once daily has been shown to be well tolerated and effective at inducing both clinical and endoscopic remission with symptom resolution in patients with mild-to-moderate UC after 8 weeks of therapy. To evaluate the potential efficacy and safety of long term therapy on the maintenance of remission with budesonide MMX, a Phase III, 12-month placebo (placebo)-controlled safety and extended use study was conducted.

Efficacy Analysis

To evaluate the efficacy of budesonide MMX 6 mg tablets having the composition as in Example 2B, for up to 12 months for the maintenance of clinical remission (i.e. rectal bleeding (RB)=−0 and stool frequency (SF)=0 based on UCDAI score), 122 patients, who were in clinical and endoscopic remission after two Phase 3 studies or an Open Label study, were randomized (1:1) to budesonide MMX 6 mg QD or placebo. Exploratory efficacy evaluation included the portion of patients in clinical remission (primary endpoint) after 1, 3, 6, 9, 12 months and/or End of Study/Early Withdrawal Visit. Secondary endpoint was time to clinical relapse (the time in days to recurrence of RB and/or SF≧1-2 stools/day above normal for the patient).

Potentially due to insufficient statistical power, efficacy analysis did not show significant difference between budesonide MMX 6 mg and placebo for the primary endpoint (p value was greater than 0.05). The probability of clinical relapse at 12 months was reduced with budesonide MMX 6 mg compared to placebo. Moreover, the median time to clinical relapse was longer and statistically significant in intended-to-treat (ITT) population in the budesonide MMX group compared to placebo (Table 1). Treatment Emergent adverse events (AEs) were similar between treatment groups (21.0%) and placebo (21.3%).

In this 12 month extended use study, the probability of clinical relapse was less likely with budesonide MMX 6 mg QD over placebo. Budesonide MMX 6 mg prolonged the median time to clinical relapse vs. placebo. Therefore, extended use of budesonide-MMX 6 mg is an option for long term therapy for the maintenance of remission in UC patients.

TABLE 1

| | Efficacy Evaluable | | Intended-to-Treat | |
|---|---|---|---|---|
| | Analysis* | | Analysis** | |
| | budesonide | | budesonide | |
| | MMX 6 mg | placebo | MMX 6 mg | placebo |
| Probability | 40.4% | 60.1% | 40.9% | 59.7% |
| of Clinical | | | | |
| Relapse | | | | |
| p-value | 0.0546 | | 0.0224 | |
| Median Time | >1 yr, never | 182 days | >1 yr, never | 181 days |
| to Clinical | reach | | reach | |
| Relapse | | | | |

*All randomized patients who received at least one dose of a study drug, but excluding patients who were not in remission at the end of the parent study, who had normal histology at baseline in the parent study, were enrolled at a site with major GCP violations in the parent study, were withdrawn from the study because of insufficient bone density
**All randomized patients who received at least one dose of a study drug

Safety Analysis

Safety analysis included 123 Patients, who were in clinical and endoscopic remission after two Phase 3 studies or an Open Label study with budesonide MMX 9 mg tablets having the composition as in Example 2A. All patients who received at least one dose of budesonide MMX 6 mg tablets having the composition as in Example 2B, or placebo were included in the safety analysis. The patients were randomized (1:1) to budesonide MMX 6 mg QD or placebo. All patients Safety assessment was conducted at baseline, 1, 3, 6, 9, 12 months of treatment and/or End of Study/Early Withdrawal Visit.

As shown in Table 2, potential glucocorticoid effects occurred at similar rates between treatment groups. Treatment-related AEs (budesonide MMX 6 mg (21.0%) and placebo (21.3%)) were similar between groups. Mean morning cortisol levels remained within the normal range for all treatment visits. SAEs were infrequently reported (one patient (1.6%) in each group) and none were related to study drug. There were no deaths or life-threatening events during the study. This extended use study showed that the rates of potential glucocorticoid effects, TEAEs, and TREAs were similar to placebo for long term maintenance of remission therapy. Thus, budesonide MMX 6 mg administered once daily for up to 12 months was well tolerated in patients with UC and with a comparable safety profile to placebo.

TABLE 2

| | Placebo | MMX 6 mg |
|---|---|---|
| MEDRA Preferred Term | n (%) | n (%) |
| Potential Glucocorticoid Ejects | N = 61 | N = 62 |
| Overall incidence | 7 (11.5) | 9 (14.5) |
| Moon face | 3 (4.9) | 3 (4.8) |
| Striae rubrae | 0 | 0 |
| Flushing | 1 (1.6) | 1 (1.6) |
| Fluid retention | 1 (1.6) | 1 (1.6) |
| Mood changes | 2 (3.3) | 4 (6.5) |
| Sleep changes | 3 (4.9) | 3 (4.8) |
| Insomnia | 4 (6.6) | 4 (6.5) |
| Acne | 0 | 3 (4.8) |
| Hirsutism | 0 | 3 (4.8) |
| Treatment Emergent AEs (TEAE) ≧ 5% | N = 61 | N = 62 |
| Patients with any TEAEs | 44. (72.1) | 40 (64.5) |
| Colitis ulcerative | 16 (26.2) | 11 (17.7) |
| Abdominal pain | 5 (8.2) | 6 (9.7) |
| Headache | 2 (3.3) | 4 (6.5) |
| Osteopenia | 5 (8.2) | 1 (1.6) |
| Frequent bowel movements | 1 (1.6) | 4 (6.5) |
| Haematochezia | 1 (1.6) | 4 (6.5) |
| Constipation | 0 | 4 (6.5) |
| Treatment-related AEs (TRAE) ≧ 2% | N = 61 | N = 62 |
| Patients With Any Treatment-related AE | 13 (21.3) | 13 (21.0) |
| Colitis Ulcerative | 3 (4.9) | 4 (6.5) |
| Osteopenia | 4 (6.6) | 1 (1.6) |
| Cushingoid | 1 (1.6) | 3 (4.8) |
| Abdominal Pain | 1 (1.6) | 2 (3.2) |
| Flushing | 1 (1.6) | 2 (3.2) |
| Hirsutism | 0 | 2 (3.2) |

Effect of Extended Use of Budesonide MMX on Bone Marrow Density

Although systemic corticosteroids (SCS) are effective for the short-term induction of remission in active UC, long term use can lead to unacceptable side effects such as a reduction in bone mineral density (BMD). Budesonide is a non-systemic corticosteroid (NSCS), and budesonide MMX (budesonide MMX) 9 mg tablets having the composition as in Example 2A, administered once daily has been shown to be well tolerated in patients with mild-to-moderate UC after 8 weeks of therapy. In this extended use study, BMD was evaluated after long term therapy with budesonide MMX 6 mg tablets having the composition as in Example 2B, over 12 months.

BMD was assessed at baseline and 12 months or the End of Study/Early Withdrawal Visit by dual-energy X-ray absorptiometry (DEXA) scan or alternative radiological methods if DEXA was not available. Patients were required to receive therapy for at least 6 months to be included in the BMD evaluation.

Baseline BMD scans were obtained from the 123 patients at the start of the long term study. 74 patients were eligible for inclusion in the BMD analysis (on drug for 6 months) (N=39; placebo and N=35; budesonide MMX 6 mg). At the end of the study, 66 patients had both baseline and end-of-study BMD scan for comparison.

As shown in Table 3, normal BMD was observed in 74% (placebo) and 78% (budesonide MMX 6 mg) at 12 months. BMD remained unchanged compared to baseline in 77% (placebo) and 86% (budesonide MMX 6 mg). Worsening of BMD was observed in 3 pts (7.7%) in placebo and 2 pts (5.7%) in budesonide MMX 6 mg. (Table 3). Overall, there were no clinically important differences between placebo and budesonide MMX 6 mg at the end of the study. There were no bone fractures reported by either group during the study. Thus, the effect of extended use of budesonide MMX 6 mg on bone mineral density appears to be comparable to placebo.

TABLE 3

| Bone Mineral Density Evaluation at | Placebo | MMX 6 mg |
|---|---|---|
| 12 months | n (%) | n (%) |
| All scan methods | 39 | 35 |
| Normal | 29 (74.4) | 27 (77.1) |
| Abnormal | 5 (12.8) | 5 (14.3) |
| Not Done | 2 (5.1) | 0 |
| Missing | 3 (7.7) | 3 (8.6) |
| DEXA Scan | 35 | 29 |
| Normal (T-score > −1) | 27 (77.1) | 21 (72.4) |
| Abnormal (T-score ≦ −1) | 5 (14.3) | 5 (17.2) |
| Osteopenia (T-score > −2.5 and ≦ −1) | 5 (100.0) | 5 (100.0) |
| Osteoporosis (T-score ≦ −2.5) | 0 | 0 |
| Missing | 3 (8.6) | 3 (10.3) |
| Other Scan methods | 4 | 6 |
| Normal | 2 (50.0) | 6 (100.0) |
| Abnormal | 0 | 0 |
| Not Done | 2 (50.0) | 0 |
| Change from baseline to last visit | 39 | 35 |
| Improved | 1 (2.6) | 0 |
| Unmodified | 30 (76.9) | 30 (85.7) |
| Worsened | 3 (7.7) | 2 (5.7) |
| Missing | 5 (12.8) | 3 (8.6) |

Effect of Extended Use of Budesonide MMX on the Hypothalamic-Pituitary-Adrenal (HPA) Axis

Although systemic corticosteroids (SCS) are effective for the short-term induction of remission in active ulcerative colitis (UC), long term use can lead to suppression of adrenal gland function resulting in reduction of circulating cortisol. Significant long term adrenal suppression may cause an adrenal crisis during acute illness, trauma, surgery, or other stress. In the 12 month study, cortisol levels and adrenocorticotropic hormone stimulation tests were evaluated during extended therapy with budesonide MMX 6 mg tablets having the composition as in Example 2B.

To assess adrenal gland function, morning plasma cortisol (MPC) levels and safety assessments were conducted at baseline, 1, 3, 6, 9, and 12 months and/or End of Study/Early Withdrawal Visit. adrenocorticotropic hormone stimulation test was performed at 12 months and/or End of Study/Early Withdrawal Visit.

At each visit, mean MPC values remained within normal limits (138 to 690 nmol/L) for both treatment groups. As shown in Table 4, a slight reduction of MPC was observed within 1 month of budesonide MMX therapy but remained within normal limits (138 to 690 nmol/L) and showed no further decline over 12 months. Mean morning cortisol levels at month 12 (nmol/L±SD) were 287.4±160.9 for budesonide MMX 6 mg and 359.7±113.4 for placebo, which were within normal limits. At Month 12/Final Visit, adrenocorticotropic hormone stimulation test showed more patients had normal values in placebo [82% (28/34)] than the budesonide MMX 6 mg group [70% (23/33)](p=0.22). This is similar to other budesonide formulations approved for the treatment of IBD, as reported in the literature. Potential glucocorticoid effects (11.5% placebo and 14.5% budesonide MMX) and treatment-related AE (21.3% placebo and 21.0% budesonide MMX) did not differ between treatment groups. No clinically significant trends in AE (including acute adrenal failure) were observed in the study. Thus, budesonide MMX 6 mg was well tolerated and the gluocorticoid effects are compared to placebo.

TABLE 4

| | Placebo | MMX 6 mg |
|---|---|---|
| Baseline, N | 59 | 59 |
| Mean ± SD (nmol/L) | 287.3 ± 153.5 | 244.6 ± 141.9 |
| Month 1, N | 45 | 49 |
| Mean ± SD (nmol/L) | 287.3 ± 98.2 | 224.7 ± 132.2 |
| Month 3, N | 40 | 45 |
| Mean ± SD (nmol/L) | 309.6 ± 96.8 | 223.4 ± 158.1 |
| Month 6, N | 35 | 31 |
| Mean ± SD (nmol/L) | 337.3 ± 124.8 | 290.6 ± 134.7 |
| Month 9, N | 30 | 29 |
| Mean ± SD (nmol/L) | 345.6 ± 116.4 | 284.1 ± 108.1 |
| Month 12- completers^{a}, N | 21 | 22 |
| Mean ± SD (nmol/L) | 348.0 ± 121.7 | 310.3 ± 141.7 |
| Changes from baseline | 64.7 ± 205.4 | 48.0 ± 129.4 |
| Month 12- all patients^{b}, N | 41 | 39 |
| Mean ± SD (nmol/L) | 359.7 ± 113.4 | 287.4 ± 160.9 |
| Changes from baseline | 74.7 ± 195.0 | 50.4 ± 174.9 |

^{a}Includes data from only those patients who completed 12 months of study drug. Patients who completed 12 months of study drug were on drug up to the time of the Final Visit.
^{b}Includes data from the Final Visit for all patients, including both those patients who completed 12 months of study drug, and those who withdrew early.
*Normal limits are 138 to 690 nmol/L.

### Example 6

### Budesonide MMX® for Add-on Therapy in Patients with Ulcerative Colitis (UC)

To establish the incremental benefit of budesonide MMX when added to current oral therapy, a randomized, double blind, placebo-controlled Phase 3b clinical study of budesonide MMX 9 mg tablets having the composition as in Example 2a, is conducted in patients with mild or moderate active ulcerative colitis (UC) who are not adequately controlled on background oral 5-ASA therapy.

The Phase 3b study evaluates patients with mild to moderate active ulcerative colitis who continue using their current 5-ASA treatment regimen and for an 8 week period add either budesonide MMX 9 mg, or placebo administered once daily. Approximately 500 patients are enrolled, with 250 in each treatment arm. The patients have been on a therapeutic dose of their oral 5-ASA (defined in this study as mesalamine ≧2.4 g/day, or equivalent dose of another approved 5-ASA (Table 5)) for a minimum of 6 weeks prior to randomization, and present with signs and symptoms of active, mild to moderate UC (UCDAI score ≧4 and ≦10 with a mucosal appearance score ≧1) in spite of their background therapy. The study compares the two treatment groups over 56 days (8 weeks). Patients remain on the same preparation and dosage strength of their oral 5-ASA for the duration of the study. Eligible patients are randomized to one of the following two treatment arms: 1. Budesonide MMX 9 mg (one tablet); 2. Placebo (tablet indistinguishable from budesonide MMX 9 mg tablet). The assigned study drugs are taken each morning after breakfast. Six visits to the clinical center are scheduled: one at Screening, four during the double-blind treatment period (Day 1, Day 14, Day 28, and Day 56), and one Safety Follow-up Visit 28 days (4 weeks) after the Day 56 visit. At Screening and Visit 5 (Day 56), patients are required to undergo a flexible sigmoidoscopy (or colonoscopy, if clinically indicated) with one photograph and three mucosal biopsies taken from the most severely affected region(s) of the colon visualized during the endoscopy procedure. Patients who are withdrawn early from the study before Day 56 are required to visit the study center as soon as possible so that the final assessments can be conducted.

The primary endpoint of the study is remission at week 8, defined as an Ulcerative Colitis Disease Activity Index (UCDAI) score of less than or equal to 1, with a zero score for rectal bleeding, stool frequency and mucosal appearance.

Patients receiving 9 mg budesonide MMX will experience a higher rate of remission of UC than patients receiving placebo.

TABLE 5
Acceptable Background 5-ASA Formulations
and Minimum Required Daily Doses

| | | Minimum Required |
|---|---|---|
| Brand Name | Generic Name | Daily Dose |
| ASACOL ®, ASACOL ® HD, | mesalamine | ≧2.4 g |
| LIALDA ®, PENTASA ®, | | |
| DELZICOL ™ | | |
| AZULFIDINE ® | sulfasalazine | ≧4.0 g |
| DIPENTUM ® | olsalazine | ≧2.0 g |
| COLAZAL ®, COLAZIDE ® | balsalazide | ≧6.75 g |

### Example 7

### Induction of Remission with Budesonide MMX® (9 Mg) Tablets in Patients with Active, Mild to Moderate Ulcerative Colitis: A Multicenter, Open-Label Efficacy and Safety Study

To assess the safety and efficacy of 8 weeks of open-label treatment with oral budesonide MMX 9 mg extended-release tablets having the composition as in Example 2A, in patients with active, mild to moderate UC, who failed to achieve clinical and endoscopic remission in a previous Phase 3, 8-week induction study, patients from a previous Phase 3 study (Parent Study) who completed 8 weeks of induction therapy in any treatment group (budesonide MMX 9 mg; budesonide MMX 6 mg; Asacol® [mesalamine]2400 mg; or placebo), and who were not in remission were eligible to enroll in the present study to receive an 8 week treatment of open label budesonide MMX 9 mg. Hence, patients who received prior budesonide MMX therapy (9 mg or 6 mg) in the previous Phase 3 study would receive an additional 8 weeks of budesonide MMX 9 mg, therapy for a total duration of 16 weeks.

The primary endpoint was induction of clinical and endoscopic remission, defined by strict criteria with a UCDAI score ≦1 after 8 weeks, with scores of 0 for rectal bleeding and stool frequency, no mucosal friability after colonoscopy, and ≧1-point reduction from baseline in the endoscopic index score. Clinical improvement (≧3 point reduction in UCDAI), endoscopic improvement (≧1 point reduction in the UCDAI mucosal appearance subscore), and symptom resolution (score of 0 for rectal bleeding and stool frequency from UCDAI) were also evaluated at the end of the treatment.

The safety variables were adverse events (AEs), laboratory test results, urinalysis, vital signs, and physical examination findings. In particular, plasma cortisol levels and potential glucocorticoid-related effects were clinically relevant parameters for this evaluation since patients previously randomized to budesonide MMX 6 or 9 mg in the Parent Study would receive budesonide MMX therapy for up to 16 weeks. All endpoints were summarized using descriptive statistics. Results were presented as overall rates of remission, clinical improvement, endoscopic improvement, and symptom resolution.

Sixty patients were administered dosages of 9 mg budesonide MMX. Fifty-two (86.7%) completed the study. Overall rates of clinical and endoscopic remission, clinical improvement, endoscopic improvement, and symptom resolution were 25.0%, 26.7%, 40.0%, and 33.3%, respectively.

Mean morning plasma cortisol (MPC) levels remained within normal limits (138 to 690 nmol/L) regardless of prior therapy (228.1 nmol/L and 167.4 nmol/L at Baseline and Week 8/Final Visit, respectively). Among the 28 patients previously exposed to 8 weeks of budesonide MMX treatment, the additional 8 weeks of open label budesonide MMX 9 mg showed a minimal decrease in MPC relative to baseline (mean A=−4.0 nmol/L, SD=149.47).

Overall frequencies of Treatment Emergent AEs (TEAEs) were similar across treatment groups. The most commonly reported TEAEs (≦5%) were blood cortisol decreased, urinary tract infection, and Cushingoid. Potential glucocorticoid-related effects (Table 6) and Treatment-related AEs were infrequent and occurred to a similar extent by treatment groups (Table 7).

About 25% of patients achieved clinical and endoscopic remission and 33.3% achieved complete symptom resolution with an 8 weeks open label budesonide MMX 9 mg after initial treatment allocation in a placebo controlled trial failed to induce remission. An 8 week course of budesonide MMX 9 mg in patients previously exposed to budesonide MMX (9 mg or 6 mg) treatment does not significantly affect MPC at Week 16/Final Visit. Thus, therapy with budesonide MMX for up to 16 consecutive weeks was well-tolerated.

TABLE 6

| | Original Parent Study Treatment Groups | | | |
|---|---|---|---|---|
| | | budesonide | budesonide | |
| | placebo | MMX 9 mg* | MMX 6 mg* | Asacol |
| | (N = 12) | (N = 12) | (N = 16) | (N = 20) |
| | n (%) | n (%) | n (%) | n (%) |
| Overall | 1 (8.3) | 0 (0.0) | 2 (12.5) | 2 (10.0) |
| Moon face | 1 (8.3) | 0 (0.0) | 1 (6.3) | 1 (5.0) |
| Striae Rubrae | 0 (0.0) | 0 (0.0) | 0 (0.0) | 0 (0.0) |
| Flushing | 0 (0.0) | 0 (0.0) | 0 (0.0) | 0 (0.0) |
| Fluid Retention | 0 (0.0) | 0 (0.0) | 1 (6.3) | 0 (0.0) |
| Moon Changes | 0 (0.0) | 0 (0.0) | 0 (0.0) | 0 (0.0) |
| Sleep Changes | 0 (0.0) | 0 (0.0) | 0 (0.0) | 0 (0.0) |
| Insomnia | 0 (0.0) | 0 (0.0) | 0 (0.0) | 0 (0.0) |
| Acne | 1 (8.3) | 0 (0.0) | 0 (0.0) | 0 (0.0) |
| Hirsutism | 0 (0.0) | 0 (0.0) | 0 (0.0) | 0 (0.0) |

TABLE 7

| | Original Parent Study Treatment Groups | | | |
|---|---|---|---|---|
| | | budesonide | budesonide | |
| | placebo | MMX 9 mg* | MMX 6 mg* | Asacol |
| | (N = 12) | (N = 12) | (N = 16) | (N = 20) |
| | n (%) | n (%) | n (%) | n (%) |
| Any TEAEs | 8 (66.7) | 6 (50.0) | 7 (43.8) | 12 (60.0) |
| Treatment- | 2 (16.7) | 1 (8.3) | 2 (12.5) | 3 (12.5) |
| related AEs | | | | |
| Blood cortisol | 2 (16.7) | 1 (8.3) | 0 (0.0) | 1 (5.0) |
| decrease | | | | |
| Cushingoid | 0 (0.0) | 0 (0.0) | 1 (6.3) | 1 (5.0) |
| Pyrexia | 0 (0.0) | 0 (0.0) | 0 (0.0) | 1 (5.0) |
| Fluid retention | 0 (0.0) | 0 (0.0) | 1 (6.3) | 0 (0.0) |
| Discontinuations | 0 (0.0) | 0 (0.0) | 1 (6.3) | 1 (5.0) |
| due to TEAEs | | | | |

### Example 8

### Induction of Remission with Budesonide MMX® (9 Mg) Tablets in Patients with Active, Mild to Moderate Ulcerative Colitis

Two similarly-designed, randomized, double-blind, placebo-controlled studies were conducted in a total of 970 adult patients with active, mild to moderate ulcerative colitis (UC) which was defined as an Ulcerative Colitis Disease Activity Index (UCDAI of ≧4 and ≦10). Eight-hundred ninety-nine of these patients had histology consistent with active UC; this was considered the primary analysis population. UCDAI is a four-component scale (total score of 0 to 12) that encompasses the clinical assessments of stool frequency, rectal bleeding, mucosal appearance and physician's rating of disease activity (score of 0 to 3 for each of the components).

The baseline median UCDAI score in both studies was 7.

In Study 1, 56% of patients were male, and the median age was 42 years. In Study 2, 57% of patients were male, and the median age was 44 years. In Study 1, 50% of patients were Caucasian, 7% were African American, and 34% were Asian. In Study 2, more than 99% were Caucasian.

Both studies compared 9 mg and 6 mg budesonide MMX, having compositions as in Examples 2A and 2B respectively, with placebo and included an active reference arm (a mesalamine 2.4 g in Study 1; and a budesonide* 9 mg not approved for the treatment of UC in Study 2). The primary endpoint was induction of remission after 8 weeks of treatment. Remission was defined as a UCDAI score of ≦1, with subscores of 0 for rectal bleeding, stool frequency, and mucosal appearance and with a ≧1 point reduction in an endoscopy-only score.

In both studies, 9 mg extended release tablets having the composition as in Example A demonstrated superiority to placebo in inducing remission (Table 8).

TABLE 8
Induction of Remission in Studies 1 and 2

| | Study 1 | Study 2 |
|---|---|---|
| Treatment Group | n/N (%) | n/N (%) |
| 9 mg budesonide | 22/123 (17.9) | 19/109 (17.4) |
| 6 mg budesonide | 16/121 (13.2) | 9/109 (8.3) |
| Reference Arm | 15/124 (12.1) | 13/103 (12.6) |
| Placebo | 9/121 (7.4) | 4/89 (4.5) |
| Treatment difference between | 10.4% (2.2%. 18.7%) | 12.9% (4.6%, 21.3%) |
| 9 mg budesonide tablet | | |
| and Placebo (95% CI)¹ | | |

CI = Confidence Interval
¹p < 0.025 for budesonide 9 mg vs. placebo in both Studies 1 and 2 based on the Chi-square test (alpha = 0.025)

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating an intestinal disease presenting at least one inflammatory component and/or maintaining remission of an intestinal disease presenting at least one inflammatory component in a patient, previously or simultaneously administered a first composition comprising at least a compound for treating said disease comprising administering to said patient, said second composition comprising:
(1) a tablet core comprising:
a) budesonide in an amount effective for treating or maintaining remission of an intestinal disease presenting at least one inflammatory component,
b) at least one lipophilic excipient;
c) at least one amphiphilic excipient; and
d) at least one hydrophilic excipient; and
(2) a coating on said tablet core, said coating comprising a gastro-resistant film.

2. The method according to claim 1, wherein the first composition is administered to the patient previously to the second composition.

3. The method according to claim 1, wherein the first composition is administered to the patient simultaneously with the second composition.

4. The method according to claim 1, wherein the intestinal disease presenting at least one inflammatory component is inflammatory bowel disease, such as ulceratve colitis, Crohn's disease or active mild to moderate ulcerative colitis.

5. The method according to claim 1, wherein the intestinal disease presenting at least one inflammatory component is acute diverticulitis and the maintenance is the maintenance of the acute phase of a diverticular disease.

6. The method according to claim 1, wherein the second composition comprises 1 mg to 18 mg budesonide in a single dose or a divided dose, such as 3 mg budesonide, 4.5 mg budesonide, 6 mg budesonide, 9 mg budesonide, 12 mg budesonide, 15 mg budesonide or 18 mg budesonide.

7. The method according to claim 1, wherein the second composition comprises 1 mg to 12 mg budesonide, such as 3 mg budesonide, 4.5 mg budesonide, 6 mg budesonide, 9 mg budesonide.

8. The method according to claim 6, wherein the second composition comprises 6 mg budesonide or 9 mg budesonide.

9. The method according to claim 1, wherein said at least a compound comprised in the first composition is selected from systemic corticosteroids and non-systemic corticosteroids.

10. The method according to claim 9, wherein said at least a compound comprised in the first composition is budesonide.

11. The method according to claim 10, wherein the first composition comprises 1 mg to 18 mg budesonide, such as 1 mg to 12 mg budesonide or 6 mg budesonide or 9 mg budesonide.

12. The method according to claim 9, wherein said at least a compound comprised in the first composition is 5-aminosalicylic acid (5-ASA).

13. The method according to claim 12, wherein the first composition comprises 2400 mg 5-aminosalicylic acid (5-ASA) or at least 2400 mg 5-aminosalicylic acid (5-ASA).

14. The method according to claim 1, wherein said at least a compound comprised in the first composition is selected from systemic antibiotics, topical antibiotics, sulphonamides, antinfective chemotherapeutics, antinfective compounds and motility-controlling drugs.

15. The method according to claim 1, wherein said at least a compound comprised in the first composition is selected from absorbable antibiotics, unabsorbable antibiobiotics, betalactamic antibiotics and chinolones.

16. The method according to claim 1, wherein said at least a compound comprised in the first composition is selected from ampicillin, amoxicillin, ciprofloxacin, fidaxomicin, erythromycin, paromomycine, trimethoprim-sulphamethoxazole, metronidazole, vancomycin, Bismuth salts, Bismuth derivatives, rifaximine, rifamycin SV, chloramphenicol, streptomycin, bacitracin and neomycin.

17. The method according to claim 16, wherein said at least a compound comprised in the first composition is rifamycin SV.

18. The method according to claim 17, wherein the first composition comprises 400 mg to 2000 mg rifamycin SV, such as 800 mg rifamycin SV or 1200 mg rifamycin SV or 1800 mg rifamycin SV.

19. The method according to claim 18, wherein the first composition comprises 400 mg to 2000 mg rifamycin SV and the second composition comprises 6 mg to 18 mg budesonide, such as 800 mg rifamycin SV and 6 mg budesonide, or 800 mg rifamycin SV and 9 mg budesonide, or 1200 mg rifamycin SV and 6 mg budesonide, or 1200 mg rifamycin SV and 9 mg budesonide, or 1800 mg rifamycin SV and 6 mg budesonide, or 1800 mg rifamycin SV and 9 mg budesonide.

20. The method according to claim 16, wherein said at least a compound comprised in the first composition is ciprofloxacin.

21. The method according to claim 20, wherein the first composition comprises 500 mg to 1500 mg ciprofloxacin.

22. The method according to claim 21, wherein the first composition comprises 500 mg to 1500 mg ciprofloxacin and the second composition comprises 6 mg to 18 mg budesonide, such as 500 mg ciprofloxacin and 6 mg budesonide, or 500 mg ciprofloxacin and 9 mg budesonide, or 750 mg ciprofloxacin and 6 mg budesonide, or 750 mg ciprofloxacin and 9 mg budesonide, or 1000 mg ciprofloxacin and 6 mg budesonide, or 1000 mg ciprofloxacin and 9 mg budesonide.

23. The method according to claim 1, wherein the second composition is administered for at least 4 weeks or for at least 8 weeks or for at least 12 months.

24. The method according to claim 1, wherein the patient is in need of maintaining remission of an intestinal disease presenting at least one inflammatory component.

25. The method according to claim 1, wherein
said at least one lipophilic excipient is stearic acid,
said at least one amphiphilic excipient is lecithin,
said at least one hydrophilic excipient is hydroxypropylcellulose, and
said gastro-resistant film comprises at least one methacrylic acid polymer or copolymer.

26. The method according to claim 1, wherein
(1) the tablet core comprises:
a) 3 mg, or 4.5 mg or 6 mg, or 9 mg or 12 mg or 15 mg or 18 mg budesonide,
b) stearic acid,
c) lecithin; and
d) hydroxypropylcellulose; and wherein
(2) the gastro-resistant film comprises at least one methacrylic acid polymer or copolymer.

27. The method according to claim 25, wherein said tablet core further comprises microcrystalline cellulose, lactose, silicon dioxide, and magnesium stearate.

28. The method according to claim 1, wherein the tablet core is a multi-matrix tablet core, wherein each lipophilic excipient is a lipophilic matrix-forming excipient, wherein each amphiphilic excipient is an amphiphilic matrix-forming excipient and wherein each hydrophilic excipient is a hydrophilic matrix-forming excipient.

29. The method of claim 1, wherein said first composition comprises 5-aminosalicylic acid, said second composition being in the form of a single tablet comprising 9 mg of budesonide.

30. The method according to claim 29, wherein the patient is treated with the first composition previously to the second composition.

31. The method according to claim 29, wherein said patient has a UCDAI score of greater than or equal to 4 prior to said second composition being administered to said patient.

32. The method according to claim 29, wherein said patient is experiencing an ulcerative colitis flare prior to said second composition being administered to said patient.

33. The method according to claim 29, wherein the second composition is administered for up to 8 weeks.

34. A method for maintaining remission of ulcerative colitis in a patient, comprising administering a composition in the form of a single tablet comprising 6 mg of budesonide

35. The method of claim 34, wherein said tablet is administered for up to 12 months or for up to six months.
